(19) United States
(12) Patent Application Publication
(10) Pub. No.: US 2013/0198900 A1
(43) Pub. Date: Aug. 1, 2013

(54) "THIAMINE PYROPHOSPHATE (TPP) RIBOSWITCH MUTANTS PRODUCING VITAMIN B1 ENRICHED FOOD AND FEED CROPS"

(71) Applicants: Asaph Aharoni, Rehovot (IL); Samuel Bocobza, Tel-Aviv (IL); Michal Shapira, Rehovot (IL)

(72) Inventors: Asaph Aharoni, Rehovot (IL); Samuel Bocobza, Tel-Aviv (IL); Michal Shapira, Rehovot (IL)

(73) Assignees: BEN-GURION UNIVERSITY OF THE NEGEV, Beer-Sheva (IL); YEDA RESEARCH AND DEVELOPMENT CO. LTD., Rehovot (IL)

(21) Appl. No.: 13/821,343

(22) PCT Filed: Sep. 7, 2011
(86) PCT No.: PCT/IL2011/000723

§ 371 Date: Mar. 29, 2013

Related U.S. Application Data
(60) Provisional application No. 61/380,332, filed on Sep. 7, 2010.

(51) Int. Cl. C12N 15/82

(52) U.S. Cl. CPC C12N 15/8243
USPC 800/278; 800/298; 435/252.3; 435/254.11; 435/257.2; 435/118

(57) ABSTRACT

The present invention provides bioengineered organisms producing elevated levels of thiamine and/or thiamine derivatives. Particularly, the present invention discloses that modifying TPP-responsive riboswitch results in accumulation of thiamine and/or its derivatives.

### FIELD OF THE INVENTION

The present invention relates to means and methods for increasing the biosynthesis of thiamine (vitamin B) and/or derivatives thereof by thiamine-producing organisms, particularly bacteria, fungi, algae and plants that can be used as animal feed or human food.

### BACKGROUND OF THE INVENTION

Thiamine, also known as vitamin B1 and aneurine hydrochloride, is one of the water-soluble B-complex vitamins. It is composed of a pyrimidine ring and a thiazol ring and the active form of this vitamin is thiamine pyrophosphate (TPP, also known as thiamine diphosphate). Thiamine is an essential coenzyme for citric-acid-cycle enzymes pyruvate dehydrogenase and α-ketoglutarate dehydrogenase, which catalyze the oxidative decarboxylation of pyruvate to acetyl coenzyme A (CoA) and α-ketoglutarate to succinyl CoA, respectively. In addition, TPP functions as a coenzyme for the ketose transketolase of the pentose—phosphate pathway. Due to its crucial role in these two pathways, thiamine is vital for cell energy supply in all living organisms. Bacteria, fungi and plants produce thiamine and its active form (TPP), whereas other organisms rely on thiamine supply from their diet. In humans, the recommended dietary allowance for thiamine is about 1.5 mg per day and thiamine deficiency leads to beriberi, a disease which can affect the cardiovascular system (referred to as “wet” beriberi) or the nervous system (referred to as “dry” beriberi, also known as Wernicke-Korsakoff syndrome).

Thiamine deficiency is a wide spread health problem that primarily concerns developing countries where rice is the major constituent of the diet, particularly since thiamine is lost during food processing (i.e. during grain, for example rice, flour refinery). Consequently, in order to cope with thiamine unavailability, refined-flour based products are enriched with thiamine in many countries. The process of thiamine enrichment (also called vitaminization or fortification) started in Canada during the 1930s. Eating a large variety of food in a balanced diet appears to be the best way to satisfy the daily need for thiamine. However, in developing countries where very little variation exists in the daily diet, thiamine enrichment seems indispensable. Compositions enriched with vitamins including thiamine, are also used as energy enhancers during physical activity.

Thiamine biosynthesis occurs in bacteria, some protozoans, plants and fungi. The thiazol and pyrimidine moieties are synthesized separately and then assembled to form thiamine monophosphate (TMP) by thiamine-phosphate synthase (EC 2.5.1.3). The exact biosynthetic pathways may differ among organisms. In E. coli and other enterobacteriaceae TMP may be phosphorylated to the cofactor TPP by a thiamine-phosphate kinase (TPhK, EC 2.7.4.16). In most bacteria and in eukaryotes, TMP is hydrolyzed to thiamine that may then be pyrophosphorylated to TPP by thiamine pyrophosphokinase (TPyK, EC 2.7.6.2). All organisms (thiamine producing and non-producing) can efficiently utilize all forms of thiamine (i.e. TMP, thiamine and TPP).

In Arabidopsis, three enzymes synthesize thiamine monophosphate (TMP), namely AtTH1 (Ajjawi et al., 2007, Arch Biochem Biophys. 459(1), 107-114); AtTHI1 (Machado C et al., 1996. Plant Mol Biol 31, 585-593; Belanger F et al., 1995. Plant Mol Biol 29, 809-821); and the TPP-riboswitch regulated AtTHIC (Croft M T et al., 2007. Proc Natl Acad Sci USA 104, 20770-20775; Wachter A et al., 2007. Plant Cell 19, 3437-3450; Bocobza S. et al., 2007. Genes Dev 21, 2874-2879; Kong D. et al., 2008. Cell Res 18, 566-576; Raschke M et al., 2007. Proc Natl Acad Sci USA 104, 19637-19642). TMP is subsequently dephosphorylated into thiamine (Komeda Y et al., 1988. Plant Physiol 88, 248-250), which is then pyrophosphorylated into TPP by the thiamine pyrophosphokinases (TPK), AtTPK1 and AtTPK2 (Ajjawi I et al., 2007. ibid).

A riboswitch is a region in an mRNA molecule that can directly bind a small target molecule, wherein the binding of the target affects the gene's activity. The small molecule targets include, among others, vitamins, amino acids and nucleotides, and the binding is selective through a conserved sensor domain. Upon substrate binding the conformation of a variable “expression platform” coupled to the sensor domain is changed and this can affect different modes of gene control including transcription termination, translation initiation or mRNA processing. Notably, riboswitches exert their functions without the need for protein cofactors. In most cases, they act in feedback regulation mechanisms: once the level of an end product in a metabolic pathway rises riboswitch binding occurs, triggering a repression of gene expression in the same pathway. The substrate specificity of riboswitches is extremely high, allowing them to perform their activity amid the presence of numerous related compounds. In prokaryotes, genetic control mediated by riboswitches is a prevalent phenomenon and the dozen riboswitches identified to date regulate over 3% of all bacterial genes.

Thiamine pyrophosphate (TPP)-binding riboswitches, first identified in Bacillus subtillis and Escherichia coli exist in the genomes of species belonging to most bacterial phyla (Rodionov D A et al., 2002. J Biol Chem 277, 48949-48959), algae and all plant species from the mosses to the most recently evolved angiosperms (Bocobza S et al., 2007. ibid). In bacteria, TPP binding to the riboswitch down-regulates expression of thiamine biosynthesis genes by inducing either the formation of a transcription terminator hairpin or the formation of a Shine-Dalgarno sequester hairpin (Mironov A s et al., 2002. Cell 111, 747-756; Rodionov et al., 2002, supra; Winkler W et al., 2002. Nature 419, 952-956). In fungi, Cheah et al. have demonstrated that a TPP riboswitch controls expression of the THI4 and NMTJ genes in Neurospora crassa by directing the splicing of an intron located in the 5′ untranslated region (UTR) (Cheah M T et al., 2007. Nature 447(7143), 497-500). Intron retention results in the appearance of upstream and out of frame initiation codons, whereas intron splicing generates a complete and correct open reading frame. In algae, it has been shown that addition of thiamine to cultures of the model green alga Chlamydomonas reinhardtii alters splicing of transcripts of the THI4 and THIC genes, encoding the first enzymes of the thiazole and pyrimidine branches of thiamine biosynthesis, respectively (Croft M T. et al., 2007. ibid). While the prokaryotic and fungi riboswitches are located in the 5′ UTR, a plant TPP riboswitch located in the 3′ UTR of the thiamine biosynthetic gene of Arabidopsis, THIAMINE C SYNTHASE (AtTHIC), was recently identified (Sudarsan N et al., 2003. RNA 9, 644-647). This difference in location suggests a unique mode of action for the plant riboswitch. Recently, the unique prevalent mechanism for TPP riboswitch-controlled gene expression in all flowering plants has been described, according to which TPP binding to THIC pre-mRNA engenders alternative splicing that leads to the generation of an unstable transcript, which in turn lowers TPP biosynthesis (Bocobza et al., 2007. ibid). Additionally, it was found that this mechanism is active in the whole plant kingdom from the mosses through angiosperms.

U.S. Pat. No. 6,512,164 discloses isolated nucleic acid fragment encoding a thiamine biosynthetic enzyme. Further disclosed is the construction of a chimeric gene encoding all or a substantial portion of the thiamine biosynthetic enzyme, in sense or antisense orientation, wherein expression of the chimeric gene results in production of altered levels of the thiamine biosynthetic enzyme in a transformed host cell.

U.S. Patent Application Publication No. 2006/0127993 discloses a method for producing thiamine products using a microorganism containing a mutation resulting in overproduction and release of thiamine products into the medium. Biologically pure cultures of the microorganisms and isolated polynucleotides containing the mutations are also provided.

U.S. Application Publication No. 20100184810 discloses methods and compositions related to riboswitches that control alternative splicing, particularly to a regulatable gene expression construct comprising a nucleic acid molecule encoding an RNA comprising a riboswitch operably linked to a coding region, wherein the riboswitch regulates splicing of the RNA, and wherein the riboswitch and coding region are heterologous.

There is an unmet need for, and it would be highly advantageous to have means and methods for efficient production of thiamine, particularly by organisms that can be consumed as a whole or that produce edible parts.

### SUMMARY OF THE INVENTION

The present invention answers the need for thiamine-enriched or thiamine-fortified food and/or feed, providing means and methods to elevate the contents of thiamine and/or its derivatives in thiamine-producing organisms, including bacteria, fungi, algae and plants. Plants comprising high amounts of thiamine and/or its derivatives are of particular interest, as particular plant species can be used as animal feed and others produce edible crops for animal and human consumption.

The present invention is based in part on the unexpected findings that (a) THIAMINE C SYNTHASE (THIC) is the rate limiting enzyme for thiamine biosynthesis, and (b) reducing the affinity of TPP-responsive riboswitch to TPP results in up-regulation of the THIC encoding gene and the synthesis of thiamine and derivatives thereof. The universality and significant conservation of the TPP-responsive riboswitch among different organisms enables utilizing the findings of the present invention to obtain food and feed products having significant elevated amounts of thiamine and/or thiamine derivatives.

Thus, according to one aspect, the present invention provides a thiamine-producing bioengineered organism comprising a modified TPP-responsive riboswitch having reduced affinity to TPP, wherein the organism produces elevated amounts of thiamine and/or derivatives thereof compared to a corresponding organism comprising an unmodified TPP-responsive riboswitch.

According to certain embodiments, the thiamine-producing bioengineered organism comprising the genetically modified TPP-responsive riboswitch produces elevated amounts of thiamine monophosphate. According to other embodiments, said organism comprises elevated amounts of thiamine. According to yet additional embodiments, said organism comprises elevated amounts of thiamine pyrophosphate.

The present invention further shows that the increased amount of thiamine and/or its derivatives leads to higher enzymatic activity of thiamine-requiring enzymes. This may lead to immediate use of the thiamine and/or its derivative and to reduction in their accumulation.

Thus, according to certain embodiments, the thiamine producing organism is further modified to have reduced activity of thiamine pyrophosphate producing enzyme or enzymes. The particular type of the thiamine pyrophosphate producing enzyme depended on the organism, as described herein and as is known in the art.

According to certain embodiments, the thiamine pyrophosphate producing enzyme is selected from the group consisting of thiamin phosphate kinase (TPhK) and thiamine pyrophosphokinase (TPyK). Each possibility represents a separate embodiment of the present invention. According to these embodiments, the organism produces elevated amounts of thiamine.

Inhibiting the expression or activity of the thiamin phosphate kinase or thiamine pyrophosphokinase may be achieved by various means, all of which are explicitly encompassed within the scope of present invention. According to certain embodiments, inhibiting TPhK or TPyK expression can be affected at the genomic and/or the transcript level using a variety of molecules that interfere with transcription and/or translation (e.g., antisense, siRNA, Ribozyme, or DNAzyme) of the TPhK or TPyK encoding genes. Inserting a mutation to these genes, including deletions, insertions, site specific mutations, mutations mediated by zinc-finger nucleases and the like can be also used, as long as the mutation results in down-regulation of the gene expression or in malfunction or non-function enzyme. Alternatively, expression can be inhibited at the protein level using, e.g., antagonists, enzymes that cleave the polypeptide, and the like.

According to some embodiments, the TPhK is encoded by a polynucleotide having the nucleic acid sequence set forth in SEQ ID NO:6. According to other embodiments, the TPhK comprises the amino acids sequence set forth in SEQ ID NO:5.

According to other embodiments, the TPyK is encoded by a polynucleotide having the nucleic acid sequence set forth in any one of SEQ ID NO:8, 10, 12, 14, 16 and 18. According to other embodiments, the TPyK comprises the amino acids sequence set forth in any one of SEQ ID NO:7, 9, 11, 13, 15 and 17.

According to certain embodiments, the organism is selected from the group consisting of bacteria, fungi, algae and plants. Each possibility represents a separate embodiment of the invention. According to some embodiments, the fungi and algae are edible. According to other embodiments, the plants are crop plants producing edible parts. According to typical embodiments, the plant is a grain producing (cereal) plant selected from, but not limited to, the group consisting of corn, soy, rice, wheat, barley, oat and rye.

According to certain embodiments, the TPP-responsive riboswitch is part of a THIAMINE C SYNTHASE encoding gene. The THIAMINE C SYNTHASE encoding gene can be the endogenous gene of the organism or an exogenous gene introduced to at least one cell of the organism using suitable transformation method as is known to a person skilled in the art. The exogenous THIAMINE C SYNTHASE encoding polynucleotide can be of any origin, including bacteria, fungi, algae and plants.

According to further embodiments, the organism comprises an expression cassette comprising a promoter sequence, a polynucleotide encoding THIAMINE C SYNTHASE and an untranslated polynucleotide comprising a modified riboswitch sequence having reduced affinity to TPP.

According to some embodiments, the modified riboswitch sequence is located upstream (5′) to the coding region. According to other embodiments, the modified riboswitch sequence is located downstream (3′) to the coding region. According to yet additional embodiments, the modified riboswitch sequence is located within the THIAMINE C SYNTHASE encoding sequence.

According to certain embodiments, the promoter is the organism's native THIAMINE C SYNTHASE promoter. According to other embodiments, the promoter is a heterologous promoter, which may be a constitutive promoter, an inducible promoter or a tissue specific promoter as is known to a person skilled in the art. According to some embodiments, the promoter is a tissue specific promoter. In these embodiments, when the organism is a plant, the tissue specific promoter is selected as to express the THIAMINE C SYNTHASE in the edible plant part. According to typical embodiments, the plant tissue specific promoter is selected from the group consisting of root, fruit and seeds specific promoter.

According to certain embodiments, the polynucleotide encodes an Arabidopsis THIAMINE C SYNTHASE (AtTHIC). The amino acids sequence of native AtTHIC (Accession No. NP_{—}850135) comprises the amino acids sequence set forth in SEQ ID NO:1, encoded by the polynucleotide comprising the nucleic acid sequence set forth in SEQ ID NO:2 (Accession No. NM_{—}179804).

Any introduced modification in the TPP-responsive riboswitch resulting in reduced affinity to TPP is encompassed by the present invention. It is to be explicitly understood that the modification can be introduced into the endogenous riboswitch, or an exogenous polynucleotide encoding a modified TPP-responsive riboswitch can be transformed into at least one cell of the organism.

According to some typical embodiments, the modification is a substitution of A to G at position 515 (A515G) relative to the stop codon of AtTHIC. According to these embodiments, the expression cassette comprises a polynucleotide having the nucleic acid sequence set forth in SEQ ID NO:3. The expression cassette comprises an AtTHIC promoter; AtTHIC encoding sequence and a riboswitch comprising the A515G mutation.

According to an additional aspect, the present invention provides a transgenic organism selected from the group consisting of bacterium, a fungus, an alga and a plant comprising at least one cell transformed with a polynucleotide encoding THIAMINE C SYNTHASE, wherein the transgenic organism produces elevated amounts of thiamine and/or its derivatives compared to a corresponding non-transgenic organism.

According to certain embodiments, the THIAMINE C SYNTHASE (THIC) is Arabodopsis THIAMINE C SYNTHASE (AtTHIC) or an ortholog thereof. According to some embodiments, the AtTHIC comprises the amino acids sequence set forth in SEQ ID NO:1 encoded by a polynucleotide having the nucleic acid sequence set forth in SEQ ID NO:2.

In the embodiments where the organism is a plant, any part of the modified plant, including pollen and seeds, as well as tissue cultures derived from said modified plant is also encompassed within the scope of the present invention.

The polynucleotides of the present invention and/or the expression cassettes comprising same can be incorporated into a plant transformation vector.

It is to be understood explicitly that the scope of the present invention encompasses homologs, analogs, variants and derivatives, including shorter and longer polypeptides, proteins and polynucleotides, as well as polypeptide, protein and polynucleotide analogs with one or more amino acid or nucleic acid substitution, as well as amino acid or nucleic acid derivatives, non-natural amino or nucleic acids and synthetic amino or nucleic acids as are known in the art, with the stipulation that these variants and modifications must preserve the THIAMINE C SYNTHASE activity and/or TPP-insensitive riboswitch activity.

According to yet further aspect the present invention provides a method for producing elevated amounts of thiamine and derivatives thereof by a thiamine-producing organism, the method comprising inserting at least one modification within a TPP-responsive riboswitch polynucleotide sequence, wherein the modification results in reduced affinity of the riboswitch to TPP, thereby obtaining an organism producing elevated amounts of thiamine and/or its derivatives compared to a corresponding wild type organism.

According to certain embodiments, the method further comprises inserting at least one modification in a TPhK encoding gene or a TPyK encoding gene. The particular modified gene depends on the organism type, as described herein and as is known in the art.

Methods for modifying a polynucleotide encoding riboswitch, TPhK or TPyK are known to a person skilled in the art and depend on the organism type.

In crop plants, point mutations in the thiamine riboswitch sequence can be obtained by chemical or otherwise mutagenesis and screening the mutant collections with a reverse-genetics technique, named Tilling. Zinc-finger nucleases, and transcription activator-like effectors nucleases (TALEN) may be also used to induce a specific alteration. A selected mutant plant having the desired modified riboswitch having reduced affinity to TPP does not contain any exogenous gene, and is non-transgenic. The crop yield is thus highly suitable to be consumed by animals and humans.

In crop plants, reduced activity of the TPyK enzymes can be obtained by chemical or otherwise mutagenesis and screening the mutant collections with a reverse-genetics technique, named Tilling. Zinc-finger nucleases, and TALEN may be also used to induce a specific alteration. Alternatively reduced activity of the TPyK enzyme can be obtained by approaches such as small interfering RNAs (siRNAs), micro RNAs (miRNA), trans-acting RNAs (tasi-RNAs), antisense RNAs (antRNA). A selected mutant plant having the desired modified TPyK activity may not contain any exogenous gene, and is not transgenic. The crop yield is thus highly suitable to be consumed by animals and humans.

According to yet additional aspect the present invention provides a method for producing elevated amounts of thiamine and derivatives thereof by a thiamine-producing organism, the method comprising transforming at least one organism cell with an expression cassette comprising a promoter, a polynucleotide encoding THIAMINE C SYNTHASE and an untranslated sequence comprising a modified riboswitch having reduced affinity to TPP, thereby obtaining an organism producing elevated amounts of thiamine and derivatives thereof compared to a corresponding wild type organism.

According to some embodiments, the modified THIAMINE C SYNTHASE is AtTHIC. According to these embodiments, the expression cassette comprises a polynucleotide having the nucleic acid set forth in SEQ ID NO:3.

According to other embodiments, the method further comprises inserting at least one modification in a TPhK encoding gene or in a TPyK encoding gene, according to the type of the organism.

Transformation of an organism selected from bacteria, fungi, algae and plants with a polynucleotide or an expression cassette may be performed by various means, as is known to one skilled in the art.

Common methods for plant transformation are exemplified by, but are not restricted to, Agrobacterium-mediated transformation, microprojectile bombardment, pollen mediated transfer, plant RNA virus mediated transformation, liposome mediated transformation, direct gene transfer (e.g. by microinjection) and electroporation of compact embryogenic calli. According to one embodiment, transgenic plants of the present invention are produced using Agrobacterium mediated transformation.

Other objects, features and advantages of the present invention will become clear from the following description and drawings.

### BRIEF DESCRIPTION OF THE DRAWINGS

FIG. 1 demonstrates the diurnal regulation of the riboswitch-dependant thiamine biosynthesis genes. Arabidopsis plants were grown in either short (FIG. 1A-1C, 1G-1J)) or long (FIG. 1D-1F) day conditions (light and dark periods are indicated by white and grey backgrounds, respectively). Transcript expression of the thiamin biosynthesis genes was measured by quantitative real time PCR (qPCR, n=3; Std Err Mean) FIG. 1A-1F—AtTHIC; FIG. 1G—AtTH1; FIG. 1H—AtTH11; FIG. 1I—AtTPK1; FIG. 1J—AtTPK2).

FIG. 2 shows a comparison of the AtTHIC transcript level in long day and short day. FIG. 2A shows superposition of the diurnal transcript levels of the AtTHIC gene coding region in short day (black) and long day (gray) conditions. Ratios of the cycle threshold (Ct) of the intron-retained to the intron-spliced variants in either short (FIG. 2B) or long FIG. 2C) day conditions is also demonstrated.

FIG. 3 shows the circadian expression of the AtTHIC gene (FIG. 3A) and its alternatively spliced variants (FIG. 3B-3C) resolved by qPCR (n=3; Std Err Mean) in Arabidopsis.

FIG. 4 shows a schematic description of the YELLOW FLUORESCENT PROTEIN (YFP) and RED FLUORESCENT PROTEIN(RFP) expression constructs.

FIG. 5 shows the circadian expression of AtTHIC (FIG. 5A) RFP (FIG. 5B) and YFP (FIG. 5C) observed in Arabidopsis plants harboring the double reporter gene system, resolved by qPCR (n=3; Std Err Mean). RFP expression is directed by the AtTHIC promoter, while YFP expression is controlled by the CaMV 35S promoter and is fused to the AtTHIC 3′ UTR (containing the riboswitch).

FIG. 6 shows the circadian levels of thiamine monophosphate (TMP, FIG. 6A) and thiamine pyrophosphate (TPP, FIG. 6B), observed in the aerial parts of 21 d old wt Arabidopsis plants grown in soil under short day conditions. Levels of TMP and TPP were examined by HPLC analysis (n=4; Std Err Mean; student's t-test indicates significant changes from the samples that show lowest metabolite levels: *, value<0.05; **, P-value<0.01). The expected light and dark periods are indicated by white and grey backgrounds, respectively.

FIG. 7 shows circadian expression of AtTHIC (FIG. 7A), AtTHI1 (FIG. 7A), and AtGRP7 (FIG. 7A) observed in Arabidopsis d975 mutants, CCA1 over-expressers and wild type Arabidopsis plants, resolved by qPCR (n=3; Std Err Mean).

FIG. 8 shows the circadian levels of thiamine monophosphate (TMP, FIG. 8A) and thiamine pyrophosphate (TPP, FIG. 8B) observed in the aerial parts of 21 d old d975 mutants, CCA1 over-expressers and wt (black) Arabidopsis plants. TMP and TPP levels were examined by HPLC analysis (n=4; Std Err Mean; student's t-test indicates significant changes from wt at a given time point: *, P-value<0.05; **, P-value<0.01).

FIG. 9 demonstrates the effect of the non-sense mediated decay (NMD) pathway on the expression of the AtTHIC gene (FIG. 9A) and its alternatively spliced variants (FIG. 9B-C). Transcript levels were measured in the background of upf1 and upf3 mutants of Arabidopsis affected in the NMD pathway compared to wild type, under normal and low endogenous TPP concentrations. Lowering the plant endogenous TPP levels in was obtained using 1 mM bacimethrin.

FIG. 10 is a schematic presentation of the system used to generate transgenic Arabidopsis plants deficient in riboswitch activity. An Arabidopsis mutant harboring a T-DNA insertion in the AtTHIC promoter [SALK_{—}011114] was used for transformation with two AtTHIC expression cassettes. One cassette contained the native AtTHIC 3′ UTR and the other contained a mutated AtTHIC 3′ UTR (A515G, relative to the stop codon), which renders the TPP riboswitch inactive.

FIG. 11 demonstrates the effect of TPP riboswitch deficiency on THIC gene expression and the production of thiamine and its derivatives. The transcript levels of the AtTHIC coding region (FIG. 11A) and its retained and spliced variants (FIGS. 11B and 11C, respectively) were measured by qPCR (n=3; Std Err Mean, student's t-test indicates significant changes from wt plants: *, P-value<0.05; **, P-value<0.01), in 21 d old wt and transgenic plants harboring the native or the mutated riboswitch. Independent lines of transformation are depicted by the line numbers.

FIG. 12 shows the circadian expression of the AtTHIC gene (FIG. 12 A) and its alternatively spliced variants (FIG. 12 B-C) resolved by qPCR (n=3; Std Err Mean) in transgenic plants harboring the native or the mutated riboswitch. Ratios of the Ct (cycle threshold) of the intron-retained to the intron-spliced variant, monitored during the circadian assay, are also depicted (FIG. 12D). The light and dark periods are indicated by white and grey backgrounds, respectively.

FIG. 13 shows the levels of thiamin, TPP, and total thiamin, observed in dry seeds or in the aerial parts of 21 days old Arabidopsis wild type and transgenic plants harboring the native or the mutated riboswitch, grown in soil under short day conditions. Amounts of thiamine and its derivatives were measured by HPLC analysis (n=5; Std Err Mean; student's t-test indicates significant changes from wt: *, P-value<0.05; **, P-value<0.01). Independent lines of transformation are depicted by the line numbers. FIG. 13A: TMP content in Arabidopsis aerial parts; FIG. 13B: Thiamine content in Arabidopsis aerial parts; FIG. 13C: TPP content in Arabidopsis aerial parts; FIG. 13D: total content of thiamine and its derivatives in Arabidopsis aerial parts; FIG. 13D: Thiamine content in Arabidopsis dry seeds.

FIG. 14 shows the transcript levels of the Arabidopsis thiamin biosynthetic genes AtTHI1 (FIG. 14A), AtTH1 (FIG. 14B), AtTPK1 (FIG. 14C) and AtTPK2 (FIG. 14D) in 21 d Arabidopsis transgenic plants harboring the native or the mutated riboswitch. Transcript levels were measured by qPCR experiments (n=3; Std Err Mean; student's t-test indicates significant changes: *, P-value<0.05; **, P-value<0.01).

FIG. 15 shows the transcript levels of the AtTHIC gene (detected by qPCR; n=3; Std Err Mean, FIG. 15A), and thiamin monophosphate (TMP, FIG. 15B) and thiamin pyrophosphate (TPP, FIG. 15C) levels (detected by HPLC analysis; n=4, Std Err Mean) in 21 d old wild type and transgenic Arabidopsis over-expressing the AtTHIC coding sequence grown in soil under short day conditions. Independent lines of transformation are depicted by the line numbers. Student's t-test indicates significant changes from wt: *, P-value<0.05; **, P-value<0.01.

FIG. 16 shows a scheme of metabolic pathways involving thiamin requiring enzymes.

FIG. 17 demonstrates that riboswitch deficiency results in enhanced activities of thiamin requiring enzymes and in increased carbohydrate oxidation through the TCA cycle and the pentose phosphate pathway. Activities of the thiamin requiring enzymes pyruvate dehydrogenase (PDH, FIG. 17A); 2-oxo-glutatarate dehydrogenase (2-OGDH, FIG. 17B; and transketolase (TK, FIG. 17C) were determined in 30 day old fully expanded leaves harvested in the middle of the light photoperiod. Measurements were performed using wild type and transgenic Arabidopsis plants harboring the native or the mutated riboswitch. Values are presented as means±SE of determinations using six independent biological replicates per genotype. Student's t-test indicates significant changes from wt plants: *, P-value<0.05; **, P-value<0.01.

FIG. 18 shows the evolution of ¹⁴CO₂ released from isolated leaf discs incubated with [1-¹⁴C]- (FIG. 18A), [3,4-¹⁴C]- (FIG. 18B), or [6-¹⁴C]-glucose (FIG. 18B). The ¹⁴CO₂ liberated was captured (at hourly intervals) in a KOH trap and the amount of ¹⁴CO₂ released was subsequently quantified by liquid scintillation counting. Measurements were performed using wild type and transgenic Arabidopsis plants harboring the native or the mutated riboswitch. Values are presented as means±SE of determinations using three independent biological replicates per genotype. Student's t-test indicates significant changes from wt plants: *, P-value<0.05; **, P-value<0.01.

FIG. 19 shows the ratio of ¹⁴CO₂ evolution from the C1 positions of glucose to that of the C6 position (FIG. 19A) or from the C3 and C4 positions (FIG. 19B) from the isolated leaf discs described in FIG. 18 hereinabove.

FIG. 20 shows the diurnal changes in amino acid levels measured in leaves of 30 day old wild type and transgenic Arabidopsis plants harboring the functional or the mutated riboswitch, using a colorimetric method. The data presented are means±SE of measurements from 6 individual biological replicates per genotype. Student's t-test indicates significant changes from wt plants: **, P-value<0.01. The light and dark periods are indicated by white and grey backgrounds, respectively.

FIG. 21 shows the diurnal changes in the glucose, fructose, sucrose, starch, proteins and nitrate levels (FIG. 21A-F, respectively) measured in leaves of 30 day old wild type and transgenic Arabidopsis plants harboring the functional or the mutated riboswitch, harvested for non-targeted analysis at 4 time points (start and middle of the light or dark photoperiods respectively). The data presented are log₁₀(means)±log₁₀(SE) of measurements from 6 individual biological replicates per genotype; the light period is 0-10 h and the dark period is 10-24 h. Independent lines of transformation are indicated by the number of the line.

FIG. 22 demonstrates the redirection of fluxes in core metabolism mediated by riboswitch deficiency. Discs of 10 weeks old wild type and transgenic Arabidopsis plants harboring a functional or the mutated riboswitch, were fed with ¹³C pyruvate or ¹³C glucose, and subjected to metabolic profiling by means of GC-TOF-MS. Changes in metabolite abundance and labeling is mapped on the metabolic network. Metabolites shown in a gray background are more abundant, while those in dark background are less abundant in plants deficient in riboswitch activity as compared to plants harboring a functional riboswitch and to wt plants according to a student's t-test, P-value<0.05 (n=6)]. Metabolites shown in white background are unchanged in this assay and metabolites noted in grey were not detected. Arrows represent either single or multiple steps. The increased activities observed for pyruvate dehydrogenase (PDH) and for 2-oxoglutarate dehydrogenase (2-OGDH) are represented by an upward arrow.

FIG. 23 shows isoprenoid content of transgenic Arabidopsis plants harboring either a native or a mutated TPP riboswitch, monitored by means of HPLC. Values are presented as means±SE (n=5). Student's t-test indicates significant changes: *, P-value<0.05; **, P-value<0.01).

FIG. 24 shows the effect of riboswitch deficiency on a range of photosynthetic parameters. Ten weeks old wild type and transgenic plants, harboring a functional or a mutated riboswitch were maintained at constant irradiance (0, 50, 100, 200, 400, 800, 1000 μE) for measurements of chlorophyll fluorescence yield and relative electron transport rate, which were calculated using the WinControl software. Photosynthetic rate (FIG. 24A), transpiration rate (FIG. 24B), water use efficiency (FIG. 24C), relative electron transport rate (FIG. 24D), stomatal conductance (FIG. 24E), and photosynthetic rate/stomatal conductance ratio (FIG. 24F), as a function of light intensity, are depicted. Each point is a mean±SE of values from 3 biological replicates per genotype.

FIG. 25 shows the diurnal changes in steady state levels of polar and semi-polar metabolites revealed using Gas Chromatography-Time Of Flight-MS (GC-TOF-MS). Independent transgenic lines harboring the mutated riboswitch (black; 3 transgenic lines), the native riboswitch (gray; 2 lines) harvested for non-targeted analysis at 4 time points (start and middle of the light or dark photoperiods respectively). A total of 43 compounds could be identified, among which 18 (depicted as graphs) exhibited differential levels in plants defective in riboswitch activity compared to plants harboring a functional riboswitch and to wt, at least at one time point (P-value<0.05; n=6). The data presented are log₁₀(means) of the measurements. The increased activities observed for pyruvate dehydrogenase (PDH) and for 2-oxo-glutarate dehydrogenase (2-OGDH) are represented by an upward arrow. Metabolites noted in black were detected, while those noted in grey were not. White and grey backgrounds in the graphs indicate the light and dark periods.

FIG. 26 shows the inhibitory effect of bacimethrin on thiamine biosynthesis in Arabidopsis wild type plants.

FIG. 27 shows the phenotype of the transgenic plants harboring the native or the mutated TPP riboswitch grown for 3 weeks (side pictures) or 5 weeks (middle pictures) in short day conditions.

FIG. 28 shows transmission electron microscopy (TEM) of leaves derived from 3 weeks old transgenic plants harboring the native or the mutated riboswitch.

FIG. 29 shows a model for TPP Riboswitch action as a pacesetter orchestrating central metabolism in thiamin autotrophs. The model represents multiple subcellular compartments including the mitochondria, chloroplast, nuclei and the cytosol. TPP, thiamin pyrophosphate; THIC, THIAMIN C SYNTHASE; CCA1, CIRCADIAN CLOCK ASSOClATED 1; var., variant; NMD, non-sense mediated decay; SAM, S-adeno syl-L-methionine; AIR, 5-aminoimidazole ribonucleotide; HMP, hydroxymethylpyrimidine; HMP-P, hydroxymethylpyrimidine phosphate; HMP-PP, hydroxymethylpyrimidine pyrophosphate; HET-P, 4-methyl-5-(β-hydroxyethyl)thiazole phosphate; NAD, nicotinamide adenine dinucleotide; CYS, cysteine; GLY, glycine; DXP, 1-Deoxy-D-xylulose-5-phosphate; TH1, thiamin-monophosphate pyrophosphorylase; THI1, thiazole synthase; thiamin-P, thiamin monophosphate; TPK, thiamin pyrophosphokinase; TK, transketolase; PDH, pyruvate dehydrogenase; 2-OGDH, 2-oxoglutarate dehydrogenase; PPP, pentose phosphate pathway; TCA cycle, tricarboxylic-acid cycle.

FIG. 30 shows a schematic presentation of the thiamine synthesis pathway in wild type (FIG. 30A) compared to riboswitch modified organism (FIG. 30B). Abbreviations are as in FIG. 29 hereinabove.

### DETAILED DESCRIPTION OF THE INVENTION

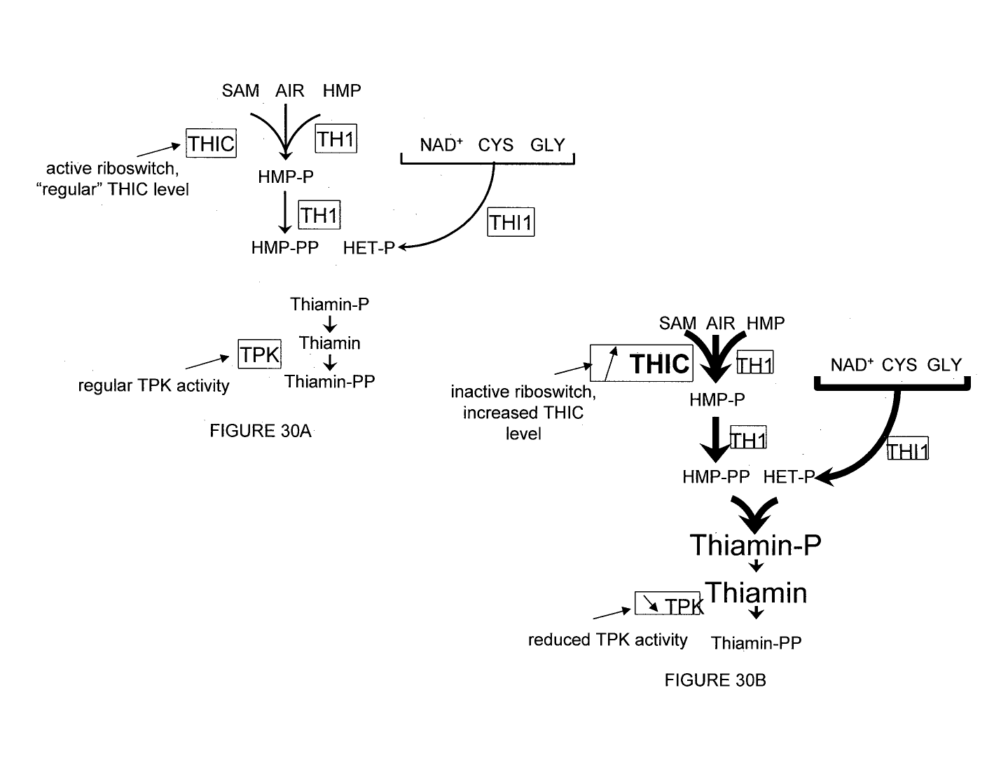

The present invention discloses thiamine-producing organisms that are so modified to produce elevated amounts of thiamine and/or thiamine derivatives compared to non-modified organisms. The present invention shows for the first time that modifying thiamine pyrophosphate (TPP) responsive riboswitch to have reduced affinity to TPP results in overexpression of thiamine synthase gene and its intron-retained variant. Furthermore, the present invention now discloses that THIAMINE C SYNTHASE is the rate-limiting enzyme in thiamine biosynthesis, and that overexpression of its coding gene results in accumulation of thiamine and/or thiamine derivatives. Schematic presentation of the native thiamine synthesis pathway is presented in FIG. 30A compared to the altered pathway according to the teachings of the present invention (FIG. 30B).

The present invention makes a significant contribution to the art by providing means for elevating the amount of the essential vitamin thiamine (vitamin B) in organisms capable of producing same. The vitamin produced can be extracted from the organism for fortifying food or feed and/or producing nutritional compositions. Additionally and preferably, the organism producing the elevated amount of thiamine is edible or produces edible parts (e.g. crop plant producing grains, fruit etc.) such that the thiamine-enriched food (or feed) can be directly consumed.

### DEFINITIONS

As used herein. The term “thiamine” (or thiamin) refers to 2-[3-[(4-amino-2-methyl-pyrimidin-5-yl)methyl]-4-methyl-thiazol-5-yl]ethanol, a water soluble, sulfur containing vitamin of the B-complex, also referred to as vitamin B or vitamin B₁.

As used herein, the term “thiamine derivatives” refers, to thiamine monophosphate (TMP) and/or thiamine pyrophosphate (TPP), either alone or in any combination.

“Elevated amount” or “elevated content” of thiamine or a derivative thereof, particularly TPP and TMP produced by an organism comprising modified TPP-responsive riboswitch depends on the type of the producing organisms. According to certain embodiments the organism is a plant, and the term refers to an increase of at least 25%, typically at least 30%, more typically 35% or more in the content of thiamine and/or its derivatives based on the fresh weight of the plant or part thereof compared to a plant comprising unmodified TPP-responsive riboswitch.

The terms “modification” and “mutation” are used herein interchangeably, to mean a change in the wild-type DNA sequence of an organism, including bacterium, fungus, alga and plant, that conveys a phenotypic change to the organism compared to the wild type organism, e.g. that allows an increase (or decrease) of thiamine or a thiamine derivative by any mechanism. The mutation may be caused in a variety of ways including one or more frame shifts, substitutions, insertions and/or deletions, inserted by any method as is known to a person skilled in the art.

The term “gene” refers to a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The term “gene” also encompasses the coding regions of a structural gene and includes sequences located adjacent to the coding region on both the 5′ and 3′ ends for a, distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

The terms “polynucleotide”, “polynucleotide sequence”, “nucleic acid sequence”, and “isolated polynucleotide” are used interchangeably herein. These terms encompass nucleotide sequences and the like. A polynucleotide may be a polymer of RNA or DNA or hybrid thereof, that is single- or double-stranded, linear or branched, and that optionally contains synthetic, non-natural or altered nucleotide bases. The terms also encompass RNA/DNA hybrids.

The term “expression cassette” as used herein refers to an artificially assembled or isolated nucleic acid molecule which includes the gene of interest. The construct may further include a marker gene which in some cases can also be a gene of interest. The expression cassette further comprising appropriate regulatory sequences, operably linked to the gene of interest. It should be appreciated that the inclusion of regulatory sequences in a construct is optional, for example, such sequences may not be required in situations where the regulatory sequences of a host cell are to be used.

According to one aspect, the present invention provides a bioengineered organism producing thiamine comprising a modified TPP-responsive riboswitch having reduced affinity to TPP, wherein the organism produces elevated amounts of thiamine and/or derivatives thereof compared to a corresponding organism comprising an unmodified TPP-responsive riboswitch.

According to certain embodiments, the thiamine-producing organism comprising the modified TPP-responsive riboswitch produces elevated amounts of thiamine monophosphate. According to other embodiments, said organism comprises elevated amounts of thiamine. According to yet additional embodiments, said organism comprises elevated amounts of thiamine pyrophosphate.

According to certain embodiments, the TPP-responsive riboswitch is part of a THIAMINE C SYNTHASE gene and/or a THI1 gene. The THIAMINE C SYNTHASE or THI1 genes can be the endogenous genes of the organism or exogenous genes introduced to at least one cell of the organism using suitable transformation method as is known to a person skilled in the art. The exogenous THIAMINE C SYNTHASE or THI1 encoding polynucleotides can be of any origin, including bacteria, fungi, algae and plants.

In prokaryotes and fungi, riboswitches are located in the 5′ untranslated region (UTR), while in plants riboswitches located within the 3′UTR has been identified. Riboswitches in algae are typically located within the coding region.

According to certain embodiments, the organism comprises an expression cassette comprising operably linked a promoter sequence, a polynucleotide encoding thiamine synthase and an untranslated sequence comprising modified riboswitch having reduced affinity to TPP.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation.

The terms “promoter element,” “promoter,” or “promoter sequence” as used herein, refer to a DNA sequence that is located upstream to the 5′ end (i.e. proceeds) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters which cause a gene to be expressed in most cell types at most times are commonly referred to as “constitutive promoters”.

According to the teachings of the present invention, the promoter can be the organism's native thiamine synthase promoteror a heterologous promoter, which may be a constitutive promoter, an induced promoter or a tissue specific promoter. According to some embodiments, the promoter is a tissue specific promoter. In these embodiments, when the organism is plant, the tissue specific promoter is selected as to express the thiamine synthase in the edible plant part. According to typical embodiments, the plant tissue specific promoter is selected from the group consisting of root and fruit promoters.

Transforming the expression cassette into at least one cell of the organism can be performed by any method as is known to a person skilled in the art.

Transformation of a cell may be stable or transient. The term “transient transformation” or “transiently transformed” refers to the introduction of one or more heterologous (or exogenous) polynucleotides into a cell in the absence of integration of the exogenous polynucleotide into the host cell's genome. Transient transformation may be detected by, for example, enzyme-linked immunosorbent assay (ELISA), which detects the presence of a polypeptide encoded by one or more of the exogenous polynucleotides. Alternatively, transient transformation may be detected by detecting the activity of a marker protein (e.g. α-glucuronidase) encoded by the exogenous polynucleotide.

In contrast, the term “stable transformation” or “stably transformed” refers to the introduction and integration of one or more heterologous (or exogenous) polynucleotides into the genome of a cell. Stable transformation of a cell may be detected by Southern blot hybridization of genomic DNA of the cell with nucleic acid sequences which are capable of binding to one or more of the exogenous polynucleotides. Alternatively, stable transformation of a cell may also be detected by enzyme activity of an integrated gene in growing tissue or by the polymerase chain reaction of genomic DNA of the cell to amplify exogenous polynucleotide sequences. The term “stable transformant” refers to a cell which has stably integrated one or more exogenous polynucleotides into the genomic or organellar DNA.

The teaching of the present invention is exemplified in plants. In this example, the expression cassette comprises operably linked promoter and polynucleotide encoding the Arabidopsis THIAMINE C SYNTHASE (AtTHIC, having SEQ ID NO:1), in which the riboswitch located within the 3′ UTR has been modified. According to certain embodiments, the modification is a nucleic acid substitution.

“Nucleic acid substitution” as used herein means a one-for-one nucleic acid replacement. According to some typical embodiments, the modification is a substitution of A to G at position 515 (A515G) relative to the stop codon of AtTHIC.

The present invention now discloses that riboswitch activity is crucial for maintaining appropriate THIC expression. As exemplified hereinbelow, THIC expression levels correlate strongly with the levels of thiamine and its derivatives. THIC over-expression, which can be obtained either by the disruption of the TPP riboswitch or by over-expression of the THIC gene, resulted in higher levels of thiamine and/or its derivatives. The universality of the TPP riboswitch, which is found in all autotrophic organisms from the most primitive bacteria to higher plants, suggests that riboswitch disruption and THIC over-expression can increase thiamine and/or its derivatives in all these organisms.

The present invention further elucidates the circadian nature of thiamine biosynthesis. The present invention now shows that the AtTHIC promoter is responsible for spatial and temporal gene control, while the AtTHIC 3′ UTR, which includes the TPP riboswitch, regulates gene expression in a TPP dose-dependent manner to degrade the AtTHIC spliced variant through the non-sense mediated decay (NMD) pathway. Specifically, it was found that the AtTHIC gene is expressed in a circadian manner, as a consequence of the activity of its promoter, to increase thiamine production during the dark period and allow the cells to cope with the mitochondrial demand for TPP during this period. Concomitantly, the TPP riboswitch ensures a proper AtTHIC expression level through differential processing of precursor-RNA 3′ terminus (Bocobza et al., 2007; ibid; Wachter et al., 2007; ibid). Interestingly, on one hand the AtTHIC promoter up-regulates AtTHIC expression during the night, while on the other hand the TPP riboswitch regulates its expression in response to TPP levels. Without wishing to be bound by any specific theory or mechanism of action, the strong diurnal correlation between thiamine levels and AtTHIC expression suggests that THIC is a major determinant of thiamine biosynthesis.

Recent reports highlight the linkage between circadian rhythm and metabolism (Fukushima A et al. 2009. Proc Natl Acad Sci USA 106, 7251-7256), as multiple clock genes were found to participate in metabolic homeostasis (Bass J. & Takahashi, J S. 2010. Science 330, 1349-54). In this regard, clock genes may direct thiamin biosynthesis, and this in turn would govern the respiration rate. Without wishing to be bound by any specific theory or mechanism of action, the potency of TPP in the regulation of carbohydrate oxidation exemplified hereinbelow may indicate that riboswitch driven thiamin biosynthesis and its circadian oscillations participate in the regulation of carbohydrate oxidation and in the light/dark metabolic transition. The molecular timer that sets the rate of starch degradation circadially has been intensively pursued during the past years, and the involvement of a circadian mechanism was reported. The circadian oscillations of TMP biosynthesis may contribute to this timer and assist the plant to anticipate dawn and sunset for optimal utilization of carbohydrate reserves during the night.

The biosynthetic pathways of thiamin and isoprenoids are tightly linked as they share a common precursor, 1-Deoxy-D-xylulose-5-phosphate (DXP). This can explain the observation that Arabidopsis plants deficient in riboswitch activity, which displayed increased thiamin production, exhibited chlorosis (similar to AtTHIC over-expressers) and reduced accumulation of isoprenoids, including carotenoids, chlorophylls and tocopherols. In addition, DXS, the enzyme that forms DXP requires TPP as a co-factor (Bouvier F et al. 1998. Plant Physiol 117, 1423-1431; Tambasco-Studart M. et al. 2005. Proc Natl Acad Sci USA 102, 13687-13692). Therefore, increased thiamin production may in turn augment DXS activity, thereby fueling the thiamin biosynthetic pathway. Interestingly, while the key gene of thiamin biosynthesis AtTHIC reaches its highest expression at sunset, the isoprenoid biosynthetic genes reach their peak of expression at dawn⁴¹. This temporal difference might prevent a direct competition between these two pathways, and allows them to share their precursors according to day/night length.

Thiamine mono-phosphate (TMP) was found to be the most abundant form in the riboswitch-modified model plant Arabidopsis. Without wishing to be bound by any theory or mechanism of action, this result implies that in plants deficient in riboswitch activity, either TMP conversion is restrained or TPP utilization is enhanced. The low level of thiamine compared to TMP and TPP levels (the most abundant form of thiamine) in wild type Arabidopsis plants suggests that pyrophosphorylation is not rate limiting in this pathway, and thiamine is efficiently pyrophosphorylated into TPP. Without wishing to be bound by any specific theory or mechanism of action, it is most probable that in the riboswitch modified plants, the overproduction of TMP, thiamine, and TPP, was concomitant with an increase in TPP usage by the thiamine requiring enzymes, leading to the accumulation of TMP over TPP.

Thus, According to certain embodiments, the thiamine producing organism is further modified to have reduced activity of thiamine-phosphate kinase (TPhK) or thiamine pyrophosphokinase (TPyK). According to these embodiments, the organism produces elevated amounts of thiamine.

Any method as is known to a person skilled in art for down regulating the expression and/or activity of TPhK or TPyK can be used. According to certain embodiments, inhibiting TPhK or TPyK expression can be affected at the genomic and/or the transcript level. According to other embodiments, expression can be inhibited at the protein level using, e.g., antagonists, enzymes that cleave the polypeptide, and the like.

Mutations can be introduced into the genes encoding the thiamin-requiring enzymes TPhK or TPyK using, for example, site-directed mutagenesis (see, e.g. Zhengm L. et al. 2004 Nucleic Acid Res. 10:32(14):e115. Such mutagenesis can be used to introduce a specific, desired amino acid insertion, deletion or substitution. Chemical mutagenesis using an agent such as Ethyl Methyl Sulfonate (EMS) can be employed to obtain a population of point mutations and screen for mutants of the TPhK or TPyK encoding gene that may become silent or down-regulated. In plants, methods relaying on introgression of genes from natural or mutated populations can be used. Cultured and wild types species are crossed repetitively such that a plant comprising a given segment of the wild or mutated genome is isolated. Certain plant species, for example Maize (corn) or snapdragon have natural transposons. These transposons are either autonomous, i.e. the transposas is located within the transposon sequence or non-autonomous, without a transposas. A skilled person can cause transposons to “jump” and create mutations. Alternatively, a nucleic acid sequence can be synthesized having random nucleotides at one or more predetermined positions to generate random amino acid substituting.

Alternatively, the expression of TPhK or TPyK can be inhibited at the post-transcriptional level, using RNA inhibitory (RNAi) molecules or antisense molecules.

RNAi refers to the introduction of homologous double stranded RNA (dsRNA) to target a specific gene product, resulting in post transcriptional silencing of that gene. This phenomenon was first reported in Caenorhabditis elegans by Guo and Kemphues (1995. Cell, 81(4):611-620) and subsequently Fire et al. (1998. Nature 391:806-811) discovered that it is the presence of dsRNA, formed from the annealing of sense and antisense strands present in the in vitro RNA preparations, that is responsible for producing the interfering activity.

The present invention thus contemplates the use of RNA interference (RNAi) to down regulate the expression of the TPhK or TPyK encoding genes to increase the level of thiamine in a thiamine-producing organism. In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs. The dsRNA used to initiate RNAi, may be isolated from native source or produced by known means, e.g., transcribed from DNA. Plasmids and vectors for generating RNAi molecules against target sequence are now readily available.

Antisense technology is the process in which an antisense RNA or DNA molecule interacts with a target sense DNA or RNA strand. A sense strand is a 5′ to 3′ mRNA molecule or DNA molecule. The complementary strand, or mirror strand, to the sense is called an antisense. When an antisense strand interacts with a sense mRNA strand, the double helix is recognized as foreign to the cell and will be degraded, resulting in reduced or absent protein production. Although DNA is already a double stranded molecule, antisense technology can be applied to it, building a triplex formation.

RNA antisense strands can be either catalytic or non-catalytic. The catalytic antisense strands, also called ribozymes, cleave the RNA molecule at specific sequences. A non-catalytic RNA antisense strand blocks further RNA processing.

Antisense modulation of the levels of TPhK or TPyK encoding genes in cells and tissues may be effected by transforming the organism cells or tissues with at least one antisense compound, including antisense DNA, antisense RNA, a ribozyme, a locked nucleic acid (LNA) and an aptamer. In some embodiments the molecules are chemically altered. In other embodiments the antisense molecule is antisense DNA or an antisense DNA analog.

Another agent capable of downregulating the expression of the TPhK or TPyK encoding genes is a DNAzyme molecule, which is capable of specifically cleaving an mRNA transcript or a DNA sequence of these genes. DNAzymes are single-stranded polynucleotides that are capable of cleaving both single- and double-stranded target sequences. A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (for review of DNAzymes, see: Khachigian, L. M. 2002. Curr Opin Mol Ther 4:119-121).

Examples of construction and amplification of synthetic, engineered DNAzymes recognizing single- and double-stranded target cleavage sites are disclosed in U.S. Pat. No. 6,326,174.

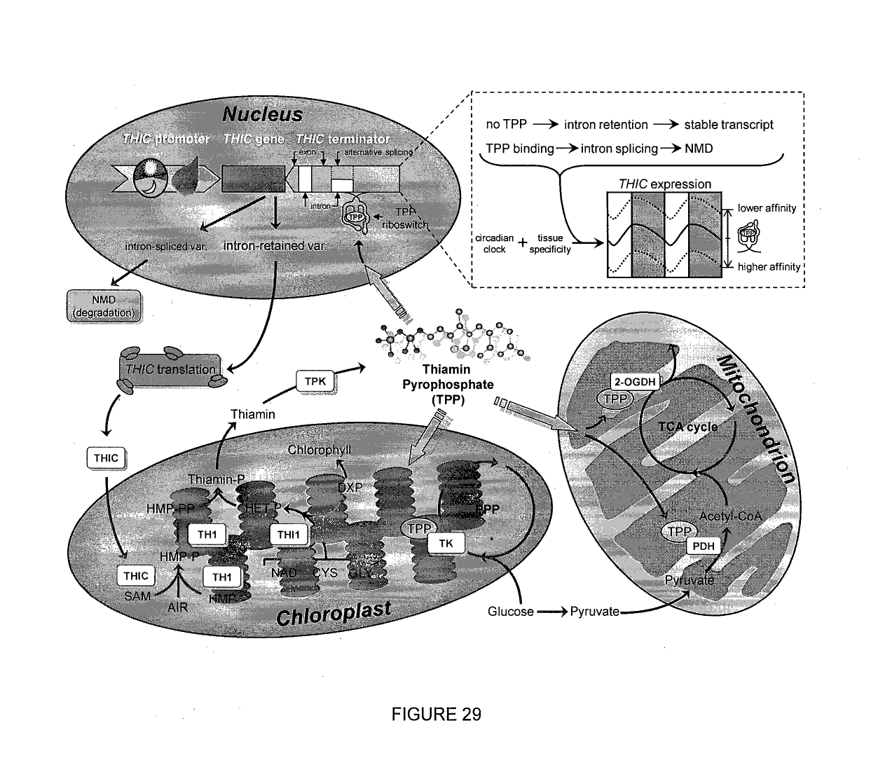

In summary, the present invention provided novel insight to the integration of a non-coding RNA mediated gene control mechanism with physiological and metabolic processes that are vital for cellular activity. Without wishing to be bound by any particular theory or mechanism of action, the model proposed (FIG. 29) is that the THIC promoter drives gene expression in a circadian manner to increase thiamin production during the dark period, while the TPP riboswitch directs the overall level of THIC expression. Consequently, high thiamin availability during the dark period enhances the activation states of the thiamin requiring enzymes, which in turn increase the carbon flux through the TCA cycle in the mitochondrion and the PPP in the chloroplast. In other words, the TPP riboswitch “senses” the TPP levels inside the nucleus and regulates thiamin biosynthesis. Thiamin pyrophosphate then reaches different subcellular compartments including the chloroplast, mitochondria and nucleus, thereby maintaining its homeostasis throughout the cell. Thus, the TPP riboswitch acts as a regulator to prevent thiamin deficiency or overdose, and together with the circadian clock, adjusts TPP availability to control the rate of carbohydrate oxidation and central metabolism diurnally. This is done in accordance with isoprenoids/chlorophyll biosynthesis, which competes for the availability of a common precursor with thiamin. Consequently, the riboswitch-directed thiamin biosynthesis tightly links the control over photosynthesis, TCA cycle and the PPP and balances primary/central metabolism and its associated downstream secondary metabolism.

The present invention exposes for the first time the regulation of thiamin biosynthesis in autotrophs, and provides means and methods to increase the thiamin content by alteration riboswitch activity. Such autotrophs, including bacteria, fungi algae and plant, particularly crop plants, having elevated levels of thiamin and/or its derivatives can be used as food to impact human populations suffering from malnutrition and thiamin deficiency.

The following non-limiting examples hereinbelow describe the means and methods for producing the transgenic plants of the present invention. Unless stated otherwise in the Examples, all recombinant DNA and RNA techniques, as well as horticultural methods, are carried out according to standard protocols as known to a person with an ordinary skill in the art.

### EXAMPLES

### Materials and Methods

### Plant Material

Arabidopsis thaliana plants (ecotype Columbia, Col-0) were grown on soil in climate rooms (22° C.; 70% humidity; 16/8 hr light/dark for long day conditions and 10/14 hr light/dark for short day conditions). The Atthi1 and Atthic mutants were obtained from the European Arabidopsis Stock Center (NASC; http://arabidopsis.info/; stock ID: N3375; salk_{—}011114 respectively). For experiments involving TPP addition, plants were grown for 14 days in petri dishes on MS media (basal salt mixture; Duchefa, Haarlem, The Netherlands) with 1% sucrose and 1% agar, to which TPP (Sigma, Cat. no. C8754, water soluble) was added up to the indicated concentration.

PCR Conditions and Oligonucleotides Used in this Study

Reactions requesting proof reading were performed with Pfu Turbo DNA polymerase (Stratagene, La Jolla, Calif.). All other reactions were performed with the GoTaq green mix (Promega, Madison, Wis.) and the respective oligonucleotides. The oligonucleotides used in this study are listed in Table 1 below.

TABLE 1
list of oligonucleotides

| SEQ ID | | |
|---|---|---|
| NO. | Oligonucleotide | Sequence |
| 19 | AtTHIC 3′UTR 5′ side Spe1 | 5′- |
| | | AATAAACTAGTAAGGTCAGTATGTTTAG |
| | | TCTGT-3′ |
| 20 | AtTHIC 3′UTR 3′ side Sal1 | 5′- |
| | | AATAAGTCGACCATAACAACGCCCAGG |
| | | ATTTCC-3′ |
| 21 | AtTHIC 3′UTR A515G Rev | 5′- |
| | | AAAAACTGCACACCCCCTGCGCAGGCA |
| | | TTACC-3′ |
| 22 | AtTHIC qPCR CDS Fwd | 5′-CCATCTTTTGAAGAATGCTTTCCT-3′ |
| 23 | AtTHIC qPCR CDS Rev | 5′-GAACACGACGAAAGGGAACTTT-3′ |
| 24 | AtTHIC qPCR Retention var. | 5′-GCCTGTTGGACTATACCTGGATAAA-3′ |
| | Fwd | |
| 25 | AtTHIC qPCR Retention var. | 5′- |
| | Rev | TGACTCAAATGAACAGACAACATAGAT |
| | | AGTT-3′ |
| 26 | AtTHIC qPCR splice var. Fwd | 5′-CTTGGTGCCTGTTGGACTATACC-3′ |
| 27 | AtTHIC qPCR splice var. Rev | 5′-TCAGGTTCAAAGGGACTTTCTCA-3′ |
| 28 | AtUBIQUITIN C qPCR Fwd | 5′-TAGCATTGATGGCTCATCCTGA-3′ |
| 29 | AtUBIQUITIN C qPCR Rev | 5′-TTGTGCCATTGAATTGAACCC-3′ |
| 30 | RFP qPCR Fwd | 5′-CTACGACGCCGAGGTCAAGA-3′ |
| 31 | RFP qPCR Rev | 5′-CGTTGTGGGAGGTGATGTCC-3′ |
| 32 | YFP qPCR Fwd | 5′-GCATCGAGCTGAAGGGCAT-3′ |
| 33 | YFP qPCR Rev | 5′-TCGGCCATGATATAGACGTTGT-3′ |
| 34 | Promoter AtTHIC Fwd | 5′-GAATTC |
| | | TTTCTTCTCCTTCTAGTGAATCAAACA-3′ |
| 35 | Promoter AtTHIC Rev | 5′-GTCGAC |
| | | AGCTGGAGACAAACGAAAATATGAATC- |
| | | 3′ |
| 36 | AtTHIC genomic Fwd | 5′- |
| | | AATAACCATGGCTGCTTCAGTACACTGT |
| | | ACCTTG-3′ |
| 37 | AtTHIC genomic Rev | 5′- |
| | | AATAGCTAGCTTATTTCTGAGCAGCTTT |
| | | GACATAG-3′ |
| 38 | AtTPK1 qPCR fwd | 5′-GCTTCAATGGGATCTCAGCAA-3′ |
| 39 | AtTPK1 qPCR Rev | 5′-CGAATCCGATTCGACTGTGAT-3′ |
| 40 | AtTPK2 qPCR Fwd | 5′-TGGGATCTCAGCAACACTGAGA-3′ |
| 41 | AtTPK2 qPCR Rev | 5′-GAAGATCCGAATCCGATTCG-3′ |
| 42 | AtTHI1 qPCR Fwd | 5′-CGCTATTGTGAGGTTGACCAGA-3′ |
| 43 | AtTHI1 qPCR Rev | 5′-CAAAAGTTGGTCCCATTCTCG-3′ |
| 44 | AtTH1 qPCR Fwd | 5′-GGCCACCATCATACAACTGAGG-3′ |
| 45 | AtTH1 qPCR Rev | 5′-ACTAACTCCATGGGACCGGC-3′ |

### Generation of DNA Constructs and Plant Transformation

For the generation of transgenic Arabidopsis, the AtTHIC 3′ UTR was amplified by PCR using Arabidopsis genomic DNA (Col0) with the oligonucleotides having SEQ ID NO:19 and SEQ ID NO:20. The mutation (A515G, starting from the stop codon) was introduced using the megaprimer-based mutagenesis strategy (Kammann M. et al., 1989. Nucleic Acids Research 17, 5404) with oligonucleotides having the nucleotide sequence as set forth in SEQ ID NOs:19, 20 and 21. The AtTHIC 3′ UTR was then fused to the YFP reporter gene and the fusion fragment was subsequently inserted downstream to the double 35S promoter of the Cauliflower Mosaic Virus (CaMV). In addition, the plasmids used to generate the transgenic plants deficient in riboswitch activity were obtained by amplifying the AtTHIC promoter with the oligonucleotides having the nucleic acid sequence set forth in SEQ ID NOs:34 and 35, and the AtTHIC genomic sequence with the oligonucleotides having the nucleic acid sequence set forth in SEQ ID NOs 36 and 37. The resulting fragment were fused adjacent to the AtTHIC 3′ UTR in the plasmids used previously. Moreover, in an additional vector, the AtTHIC promoter was amplified similarly with oligonucleotides having the nucleic acid sequence set forth in SEQ ID NOs:34 and 35 and fused to the RFP reporter gene, which was adjacent to the NOS terminator.

The cassettes were then inserted into the pBinPlus binary vector containing the kanamycin resistance gene for the selection of transformants (van Engelen F A. et al., 1995. Transgenic Res 4, 288-290), or into the pGreenII vector (http://www.pgreen.ac.uk/pGreenII), containing the Basta resistance gene for selection of transformants. Arabidopsis plants were transformed using the floral-dip method (Clough S J. and Bent A. F., 1998. Curr Opin Microbiol 10, 176-181) and kanamycin-resistant seedlings were then transferred to soil.

### Molecular Biology and Microscopy

Unless specified, all molecular biology manipulations were performed as described in Sambrook J et al., 1989. Molecular cloning: A laboratory manual. (Cold spring harbor laboratory press). Leaf samples were taken at the time point indicated, immediately frozen in liquid nitrogen and stored at −80° C. until further analysis. Extraction was performed by rapid grinding of the tissue in liquid nitrogen and immediate addition of the appropriate extraction buffer. RNA extractions were all performed using the RNeasy kit (Qiagen, Valencia, Calif.) and DNA extractions using the CTAB method (Doyle J. and Doyle J., 1987. Phytochem Bull 19, 11-15).

### Circadian and Diurnal Assays

For circadian gene expression analysis, plants were grown in soil under normal short day conditions (10/14 h of light/dark), for three weeks, followed by three days of constant light. Plant samples were harvested every four hours during the last two days of constant light. For diurnal measurements of RNA expression and metabolite levels, plants were grown in soil under normal short (10/14 h of light/dark) or long (18/6f of light dark) day conditions for 3 weeks after which samples were harvested every 4 hours during 24 hours. Gene expression was assessed by qPCR using the AtUBIQUITIN C as a control.

### Bacimethrin Treatment

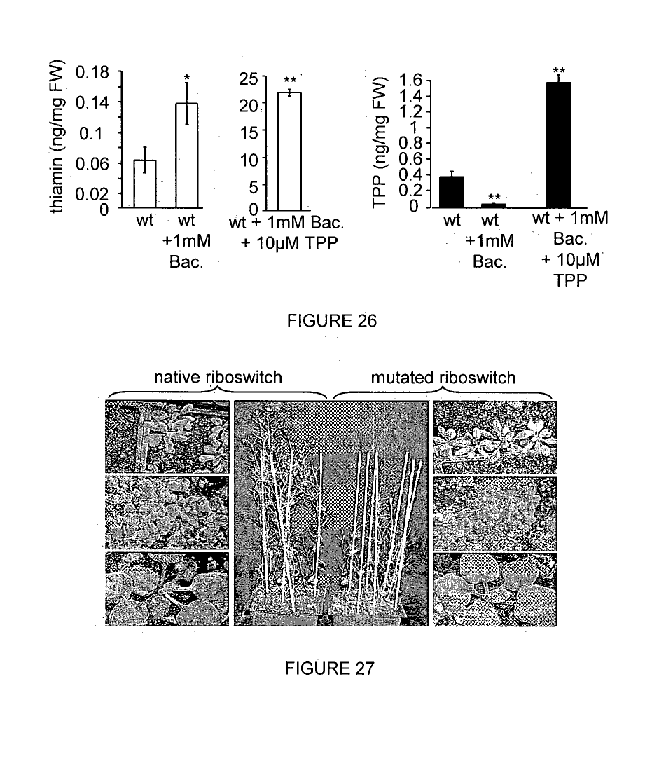

Bacimethrin is a naturally occurring thiamine anti-metabolite. It is converted to 2′-methoxy-thiamine pyrophosphate by the thiamine biosynthetic enzymes at a rate that is 6 times faster than the rate of conversion of the natural substrate HMP to thiamine pyrophosphate (Reddick J J et al., 2001. Bioorg Med Chem Lett 11, 2245-2248). Since bacimethrin is a thiamine biosynthesis inhibitor in bacteria, its potency in plants was examined. For this purpose, bacimethrin was synthesized according to Koppel et al. (Koppel S et al., 1962. Pyrimidines. X. (Antibiotics. 11) Synthesis of Bacimethrin, 2-Methoxy) It was found that addition of 1 mM bacimethrin to the growth medium increased thiamine levels but reduced TPP levels in wild type plants (FIG. 26).

### Transcriptome and Gene Expression Analysis

Transcriptome analysis was performed as described in Panikashvili et al. (Panikashvili D et al., 2010. Mol Plant 3, 563-575). Total RNA was extracted from 4 weeks old seedlings using the RNeasy Plant Mini Kit (Qiagen). cDNA was synthesized using the One-Cycle cDNA Synthesis Kit (Invitrogen). The double-stranded cDNA was purified and served as a template in the subsequent in vitro transcription reaction for complementary RNA (cRNA) amplification and biotin labeling. The biotinylated cRNA was cleaned, fragmented, and hybridized to Affymetrix ATH1 Genome Array chips. Statistical analysis of microarray data was performed using the Partek Genomics Suite (Partek Inc., St Louis, Mo.) software. CEL files (containing raw expression measurements) were imported to Partek GS. The data were pre-processed and normalized using the RMA (Robust Multichip Average) algorithm (Irizarry R et al., 2003. Biostatistics 4, 249-264). The normalized data were processed by PCA (Principal Component Analysis) and hierarchical clustering to detect batch or other random effects that may appear in case the replicates are carried out sequentially. To identify differentially expressed genes one way ANOVA analysis of variance was applied. FDR (false discovery rate) was used to correct for multiple comparisons (Benjamini Y and Hochberg Y., 1995. J. of the Royal Statistical Society. Series B (Methodological) 57, 289-300). Gene lists were created by filtering the genes based on fold change and signal above background in at least one microarray. Up-regulated genes were defined as those having a greater than or at least two-fold linear intensity. The MapMan software was used in order to create MapMan overview diagrams of the microarray data (Thimm O et al., 2004. Plant J 37, 914-939) and classify differentially expressed transcripts into functional categories.

Quantitative Real Time PCR (qPCR) gene expression analysis was performed with three biological replicates using gene/variant-specific qPCR oligonucleotide-pairs, designed with Primer Express software (Applied Biosystems). Specific oligonucleotides sequences are provided in the table 1 hereinabove. AtUBIQUITIN C (At5g25760) was used as endogenous control for all analyses. Fixed amount of DNAse-treated total RNA was reverse transcribed using AMV Reverse Transcriptase (EurX Ltd., Poland). RT-PCR reactions were tracked on an ABI 7300 instrument (Applied Biosystems) using the PlatinumR SYBR SuperMix (Invitrogen). Each sample was PCR-amplified from the same amount of cDNA template in triplicate reactions. Following an initial step in the thermal cycler for 10 min at 95° C., PCR amplification proceeded for 40 cycles of 15 s at 95° C. and 60 s at 60° C., and completed by melting curve analysis to confirm specificity of PCR products. The baseline and threshold values were adjusted according to manufacturer's instructions.

### HPLC Analysis of Thiamine Derivatives

Samples (100 mg) were harvested from three weeks old plants grown in short day conditions at the end of the light period, and immediately frozen in liquid nitrogen. The plant samples were then grinded followed by the addition of 400 μl of 0.1M HCl, and sonicated in a water bath for 30 min. The resulting extracts were centrifuged at 14,000 rpm (in a regular bench centrifuge) for 10 min. Samples of 300 μl of the supernatant were supplemented consecutively with 50 μl of freshly made 10 mM K₄Fe(CN)₆, which was dissolved in 3.7N NaOH, and 100 μl of MeOH(HPLC grade). The samples were vigorously shaken, sonicated for 5 min, and centrifuged at 14,000 rpm (on a regular bench centrifuge) for 10 min. For measurements of dry seeds, 30 mg seeds were grinded and the following ratios were used: 250 μl HCl, 150 μl of the supernatant was supplemented with 25 μl K₄Fe(CN)₆ and 50 μl MeOH. Following centrifugation, supernatants were then fractionated with a Capcell Pak NH₂ column (150 mm×4.6 mm i.d.) (Shiseido, Tokyo) using a 4:6 (v/v) solution of 100 mM potassium phosphate buffer pH=8.4 and acetonitrile as mobile phase. The HPLC analyses were performed using a Merck L7200 autosampler, a Merck L7360 column oven set at 25° C., a Merck pump Model L7100, and a Merck FL-detector L7480. A Merck D7000 interface module was used and the chromatograms were integrated using the HSM software. The flow rate was 0.5 ml/min, and the volume injected was 5 μl for all samples. Thiochrome derivatives of thiamine, TMP, and TPP were detected by fluorescence at excitation 370 nm and emission 430 nm. Different concentrations of thiamine, thiamine monophosphate (TMP) and thiamine pyrophosphate (TPP) standards were analyzed using the same extraction procedure and chromatographic conditions. Calibration curves were generated for each of the standards. For quantification of the samples, the peak areas of the samples were compared to the corresponding standard curve.

### Measurement of Respiratory and Photosynthetic Parameters

Measurements of respiratory and photosynthetic parameters were performed according to Nunes-Nesi A et al. (2007. Plant J 50, 1093-106). Estimations of the pentose phosphate pathway and TCA cycle flux on the basis of ¹⁴CO₂ evolution were carried out following incubation of leaf discs taken from 4 week old plants, in 10 mM MES-KOH, pH 6.5, containing 0.3 mM glucose supplemented with 2.32 kBq ml⁻¹ of [1-¹⁴C]-, [3,4-¹⁴C]- or [6-¹⁴C]-glucose. This was performed in the dark at the end of the light period. ¹⁴CO₂ produced was trapped in KOH and quantified by liquid scintillation counting. Notably, carbon dioxide can be released from the C₁ and C₆ positions by the action of enzymes associated with the PPP, while it can be released from the C₃₋₄ positions of glucose by enzymes associated with mitochondrial respiration (Nunes-Nesi. et al., 2005. Plant Physiol 137, 611-622). Moreover, the ratio of ¹⁴CO₂ evolution from the C₁ or the C₆ position of glucose to that from the C_{3,4} positions of glucose provides an indication of the relative rate of the TCA cycle with respect to other processes of carbohydrate oxidation (such as glycolysis and the PPP).

Fluorescence emission was measured in vivo using a PAM fluorometer (Walz; http://www.walz.com/) on 5-week-old plants maintained at fixed irradiance (0, 50, 100, 200, 400, 800, and 1000 μmol photons m⁻² sec⁻¹) for 30 min prior to measurement of chlorophyll fluorescence yield and relative ETR, which were calculated using the WinControl software package (Walz). Gas-exchange measurements were performed in a special custom-designed open system (Lytovchenko et al., 2002). The CO₂ response curves were measured at saturating irradiance with an open-flow gas exchange system (LI-COR, model LI-6400; http://www.licor.com/).

### Determination of Metabolite Levels

The levels of starch, sucrose, fructose and glucose in the leaf tissue were determined as described previously (Fernie A et al., 2001. Planta 212, 250-63). Levels of proteins, amino-acids, and nitrate were assayed as described by Sienkiewicz-Porzucek A et al. (2010. Mol Plant 3, 156-73) and Tschoep, H. et al. (2009. Plant Cell Environ 32, 300-18). All measurements were performed using 4 weeks old Arabidopsis aerial parts (50 mg fresh weight) harvested diurnally at the beginning and the middle of the light and dark periods. Additionally, metabolite profiling of 4 weeks old wild type (wt) and transgenic plants deficient in riboswitch activity, were determined from 100 mg plant extracts using a GC-TOF-MS apparatus as described previously (Koppel, S., ibid). In this experiment, metabolites from 3 independent lines of transformation harboring the defective riboswitch, two lines harboring the functional riboswitch, and wt plants were monitored. We considered as “altered” only the metabolites that differed in all 3 transgenic lines harboring the defective riboswitch from the control and wt plants.

### Measurement of Isotope Redistribution

The fate of ¹³C-labeled pyruvate and glucose was traced following feeding of isolated leaf discs from 6 weeks old transgenic Arabidopsis plants deficient in riboswitch activity compared to control and wt, grown in short day conditions, incubated in the dark at the end of the light period, since amino acids levels were most affected during this period (FIG. 20), in a solution containing 10 mM MES-KOH (pH 6.5) and 10 mM [U-¹³C]-pyruvate or 10 mM [U-¹³C]-glucose for 2 h and 4 h. Fractional enrichment of metabolite pools was determined exactly as described previously (Roessner-Tunali U. et al., 2004. Plant J 39, 668-79). Label redistribution was calculated as described by Studart-Guimarães, C. et al. (2007. Plant Physiol 145, 626-39).

### Isoprenoid Profiling

Isoprenoids content analysis was performed using an HPLC-PDA detector (Waters, http://www.waters.com) and an YMC C30 column (YMC Co. Ltd., http://www.ymc.co.jp/en/) as described by Fraser P et al. (2000. Plant J 24, 551-8). Peak areas of the compounds were determined according to the spectral characteristic

### Metabolomic Assays by Means of UPLC-qTOF-MS

UPLC-qTOF-MS analysis of four weeks old Arabidopsis plants grown in short day conditions and harvested diurnally at the beginning and the middle of the light and dark period was performed according to Malitsky S et al. (2008. Plant Physiol 148, 2021-49).

### Example 1

### Regulation of the THIC Gene in Plant Green Tissues

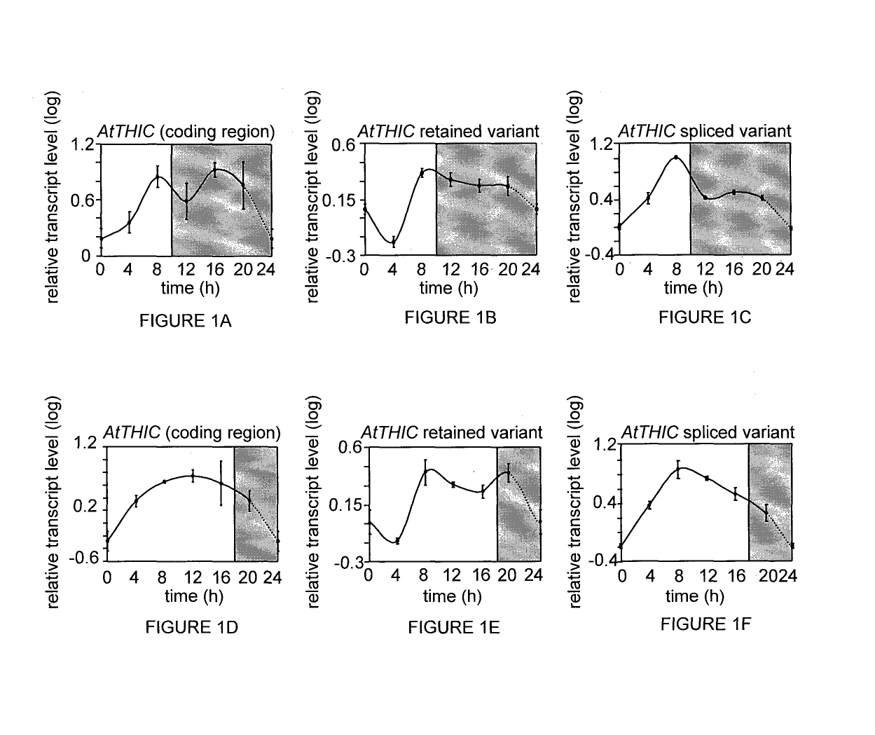

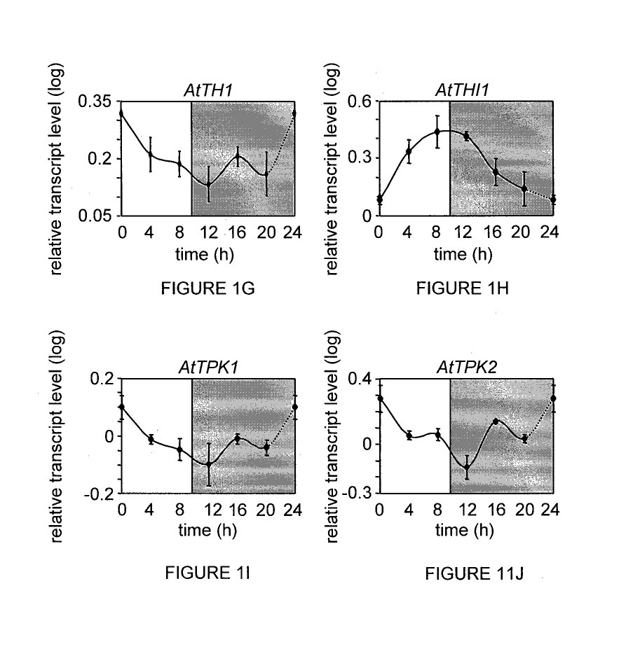

The changes in the expression of thiamine biosynthesis genes, particularly THIC gene and its splicing products, were examined under various light conditions. It was previously found that the level of the two THIC 3′ UTR splice variants is riboswitch-dependent and respond to altered cellular TPP concentrations: when the level of the TPP ligand rises, the expression of the unstable intron-spliced variant increases and the stable intron-retained variant decreases, and vice versa (Bocobza et al., 2007, ibid; Wachter A et al., 2007. ibid). The present invention now shows that all genes involved in thiamine biosynthesis appear to be regulated in a diurnal manner. It was found that the expression of the AtTH1, AtTPK1 and AtTPK2 transcripts decreases during the light period and increases during the dark period, while AtTHI1 and the AtTHIC transcripts (both the coding region and its two alternatively spliced variants) showed the opposite expression profile (FIG. 1A-1J).

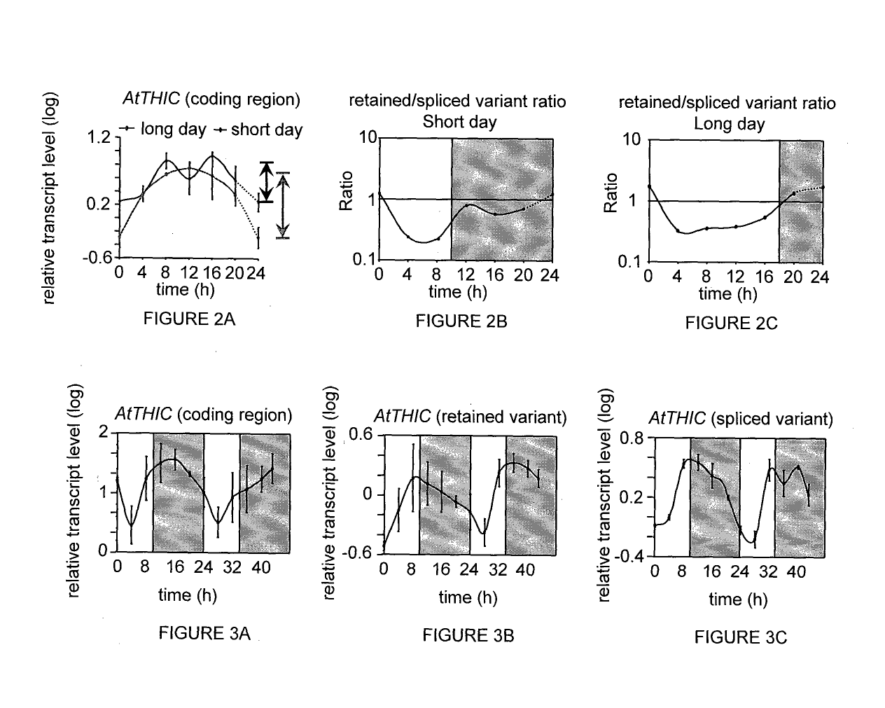

All AtTHIC transcripts displayed a similar expression profile when plants are grown either in short (10 h) or long (18 h) day conditions. However, it was also observed that the amplitude of the diurnal change in the AtTHIC transcript level is about twice larger when plants are grown in long day conditions compared to short day (FIG. 2A). Additionally, the ratio between the intron-retained and the intron-spliced variant also changed in a diurnal manner. In both long and short day conditions the expression level of the intron-spliced variant was higher than that of the intron-retained variant during the light period, while the opposite was observed during the dark period (FIG. 2B-C).

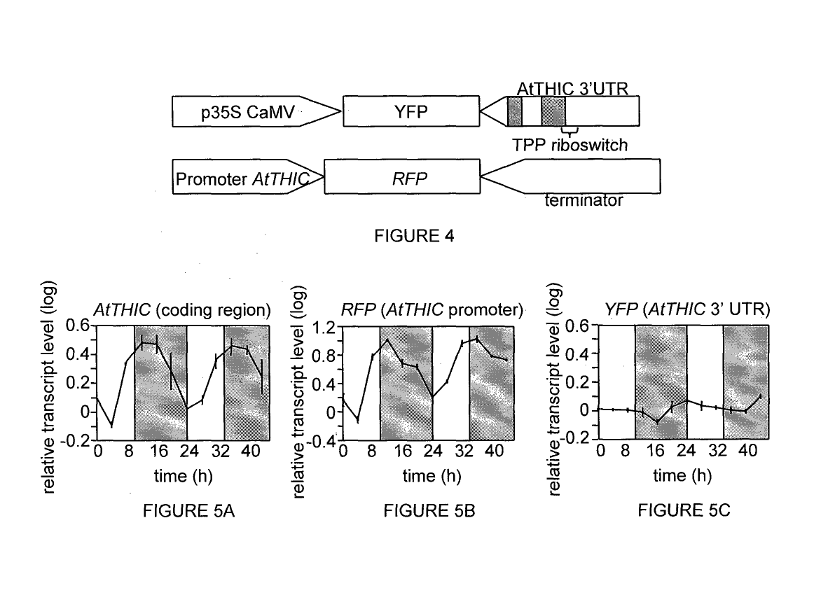

In order to determine whether the diurnal changes of AtTHIC expression are caused by the light or by the circadian clock, Arabidopsis plants were subjected to a circadian assay (see material and methods above). The AtTHIC transcript and its variants displayed circadian oscillations similar to the diurnal ones (FIG. 3), indicating the circadian regulation of AtTHIC expression. In the growth conditions used herein, the transcripts reached their highest expression level at the beginning of the dark period and their lowest expression level shortly after the beginning of the light period. These results raised the question whether the TPP riboswitch takes part in the circadian regulation of AtTHIC, or whether additional regulatory elements (e.g. the AtTHIC promoter in the 5′ region) are directly responsible for these oscillations. To separate between these two modes of gene control, a dual reporter assay was developed, allowing observing the in vivo activity of both elements. Expression of one reporter gene, YELLOW FLUORESCENT PROTEIN(YFP) was under the control of a constitutive promoter (CaMV-35S) and fused to the AtTHIC 3′ UTR. A second reporter gene, RED FLUORESCENT PROTEIN(RFP) was placed under the regulation of the native AtTHIC promoter and fused to the NOS terminator (FIG. 4). These two reporters were introduced into the Arabidopsis wild type (wt) and thi1 mutant (deficient in thiamine biosynthesis), and their expression was monitored under various conditions. Wild type plants harboring this double reporter genes system were subjected to a circadian assay. In these plants, both the AtTHIC transcript and the RFP reporter (driven by the AtTHIC promoter) displayed circadian oscillations, identical to those described above, while YFP expression (fused to the AtTHIC 3′ UTR) remained constant (FIG. 5). This result suggests that the circadian oscillations observed in AtTHIC expression are merely the consequence of promoter activity and are not the result of TPP riboswitch activity.

The expression level of the above-described reporter genes (YFP and RFP) in fully grown plants was also determined. In the wild type background plants, the AtTHIC promoter directed RFP expression in all green tissues, but not in roots, seeds, and petals, while the AtTHIC 3′ UTR represses YFP expression, probably because of the endogenous TPP levels. This result is in accordance with the finding that THIC is targeted exclusively to the chloroplast. It should be noted that in younger tissues (young leaves and buds), YFP expression appeared stronger than in older ones, but this is probably due to the higher cell density in these tissues. In order to determine if either the AtTHIC promoter and/or the 3′ UTR respond to altered TPP levels, wt plants harboring the double reporter gene system were exposed to increasing TPP concentrations. Only slight changes were observed in the reporter gene expression, indicating that endogenous TPP levels present in wt plants are sufficient to mask (at least partially) the effect of the exogenous TPP applied in the experiment. However, when these reporter genes were co-introduced into Atthi1 mutant plants, the AtTHIC promoter did not respond to TPP, while the AtTHIC 3′ UTR regulated YFP expression in a TPP dependent manner. Overall, these results suggest that the AtTHIC promoter directs gene expression in a time- and organ-specific manner but is not TPP responsive, while the AtTHIC 3′ UTR regulates gene expression in response to TPP in a dose-dependent manner.

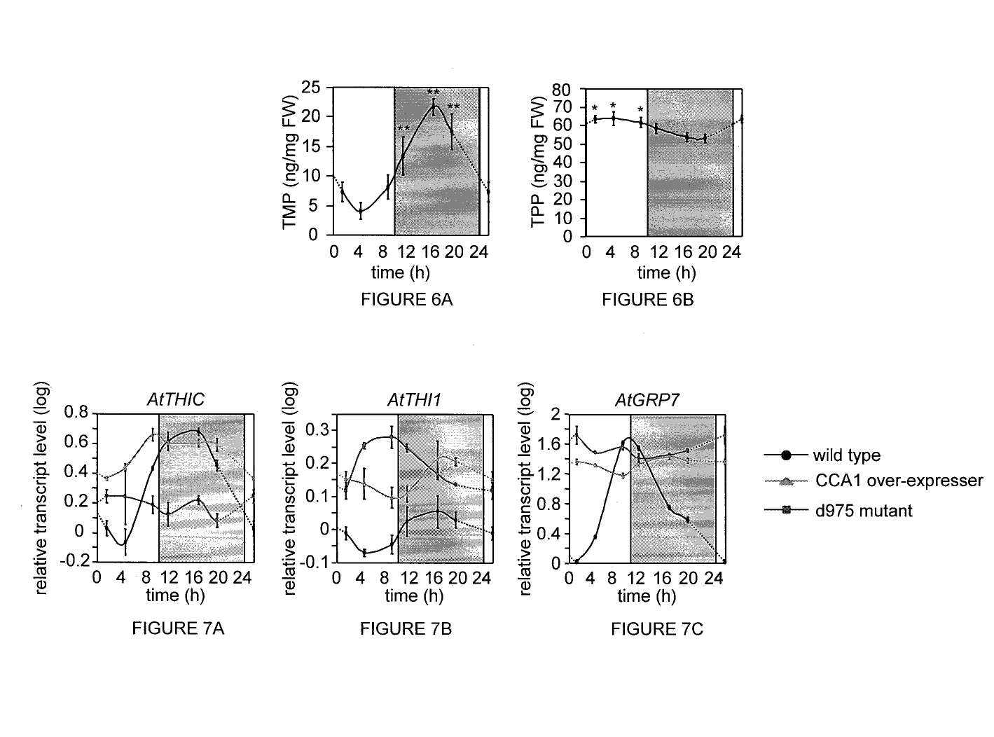

The diurnal oscillations observed in the expression of the thiamine biosynthetic genes suggested that the level of thiamine metabolites could also be modulated in a diurnal manner. Thus, the level of thiamine and its derivatives throughout the day was measured. It was found that in Arabidopsis, as observed in other organisms, the majority of thiamine is in the form of TPP, suggesting that TMP and thiamine were continuously converted to TPP. Surprisingly, it was also observed that the TMP levels displayed circadian oscillations similar to the ones observed for AtTHIC and AtTHI1 expression (FIG. 6A). Thiamine monophosphate levels were highest at the end of the light period and lowest at the end of the dark period, while TPP levels were slightly lower during the dark period than during the light period (FIG. 6B). Thiamine levels were barely detectable in this assay. Taken together, these results indicate that TMP levels oscillate in a circadian manner, most likely to supply TPP precursors during the dark period when respiration remains the only source of energy production.

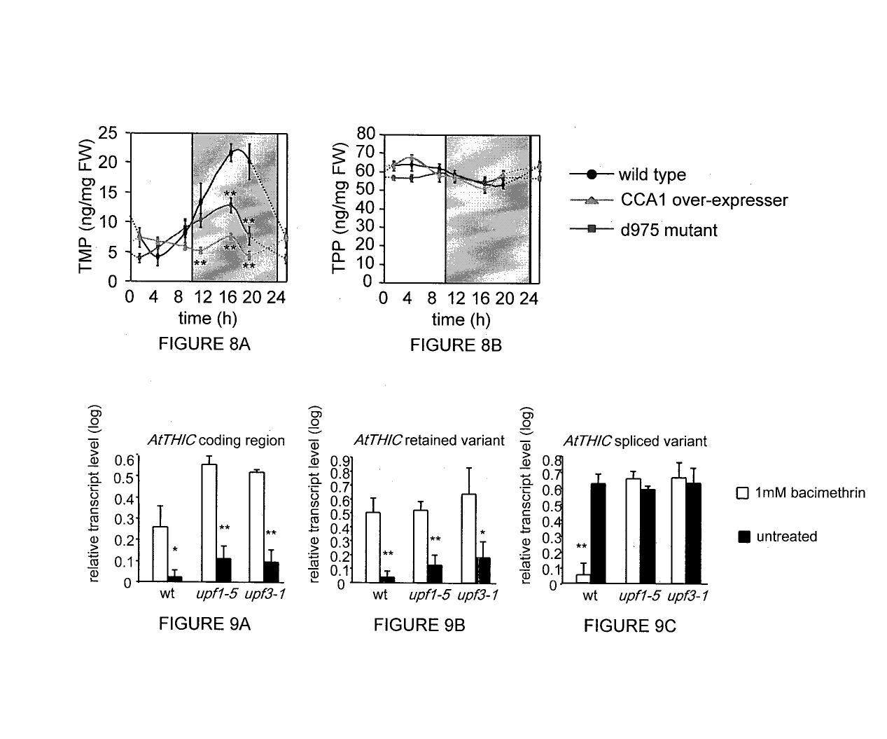

In order to determine whether the circadian oscillations described above were the consequence of the biological clock action, the expression of the thiamine biosynthetic genes was measured, as well as the levels of thiamine derivatives in transgenic plants altered in their biological clock. In this experiment, the prr9-11 prr7-10 prr5-10 triple mutant (d975) and the CCA1 over-expresser were used (Nakamichi N et al., 2009. Plant Cell Physiol 50, 447-462; and Wang Z Y & Tobin E M. 1998. Cell 93, 1207-1217, respectively). Interestingly, it was observed that in these plants, circadian expression of the AtTHIC and AtTHD genes was considerably altered as compared to wt (FIG. 7A and FIG. 7B respectively; the AtGRP7 gene (FIG. 7C) was used as a positive control). Furthermore, the circadian oscillations observed for TMP levels were also altered in these arrhythmic plants (FIG. 8A), while TPP levels were not affected (FIG. 8B). Without wishing to be bound by any specific theory or mechanism of action, these findings support the hypothesis that thiamine biosynthesis is regulated by the circadian clock.

### Example 2

### AtTHIC Regulation

Given the importance of the AtTHIC gene for thiamine biosynthesis, its mode of regulation, particularly the nature of the high turnover of the intron-spliced variant compared to the intron-retained variant was further examined. Since the spliced variant contains two introns in its 3′ UTR, its instability could be due to the activity of the non-sense mediated decay (NMD) pathway (Kertesz S et al., 2006. Nucleic Acids Res 34, 6147-6157). Thus, the level of this transcript was measured in the background of upf1 and upf3 mutants of Arabidopsis affected in the NMD pathway (Arciga-Reyes L et al., 2006. Plant J 47, 480-489). Transcript level was examined in both normal and low TPP concentrations (FIG. 9). Lowering the plant endogenous TPP levels in these experiments was obtained using 1 mM bacimethrin, an anti-metabolite that inhibits thiamine production (Reddick et al., 2001; ibid). In wt plants as well as in the upf1 and upf3 mutants, bacimethrin treatment (preventing thiamine accumulation) caused the upregulation of the total AtTHIC transcript and of its intron-retention variant (FIGS. 9A and 9B, respectively). However, while bacimethrin treatment caused the down-regulation of the intron-spliced variant in wt plants, it did not decrease the level of this variant in the upf1 and upf3 mutants (FIG. 9C). Without wishing to be bound by any specific theory or mechanism of action, these results denote that these mutants can accumulate intron-spliced transcripts when intracellular TPP concentrations are low. As shown previously, the intron-spliced variant exhibits an average decay rate of 63% per hour (Bocobza et al., 2007; ibid). Thus, the accumulation of this transcript in the upf mutants can be explained by an increase in its stability since the NMD trans-factors are missing. It should also be noticed, that due to the accumulation of the intron-spliced variant under bacimethrin treatment, the total AtTHIC transcripts are more abundant in the upf mutants. This result suggests the participation of the UPF1 and UPF3 proteins in the destabilization of the AtTHIC intron-spliced transcript, and the role of the NMD pathway in the regulation of the AtTHIC gene. Furthermore, the levels of thiamine and its derivatives in the upf1 and upf3 mutants were measured thiamine and were shown to be similar to wt. Since, in these mutants, the AtTHIC transcripts are more abundant (due to the accumulation of the intron-spliced variant) while thiamine levels are not affected, this result implies that the intron-spliced transcript is not translated into an active THIC protein.

### Example 3

### Disruption of the Plant TPP Riboswitch

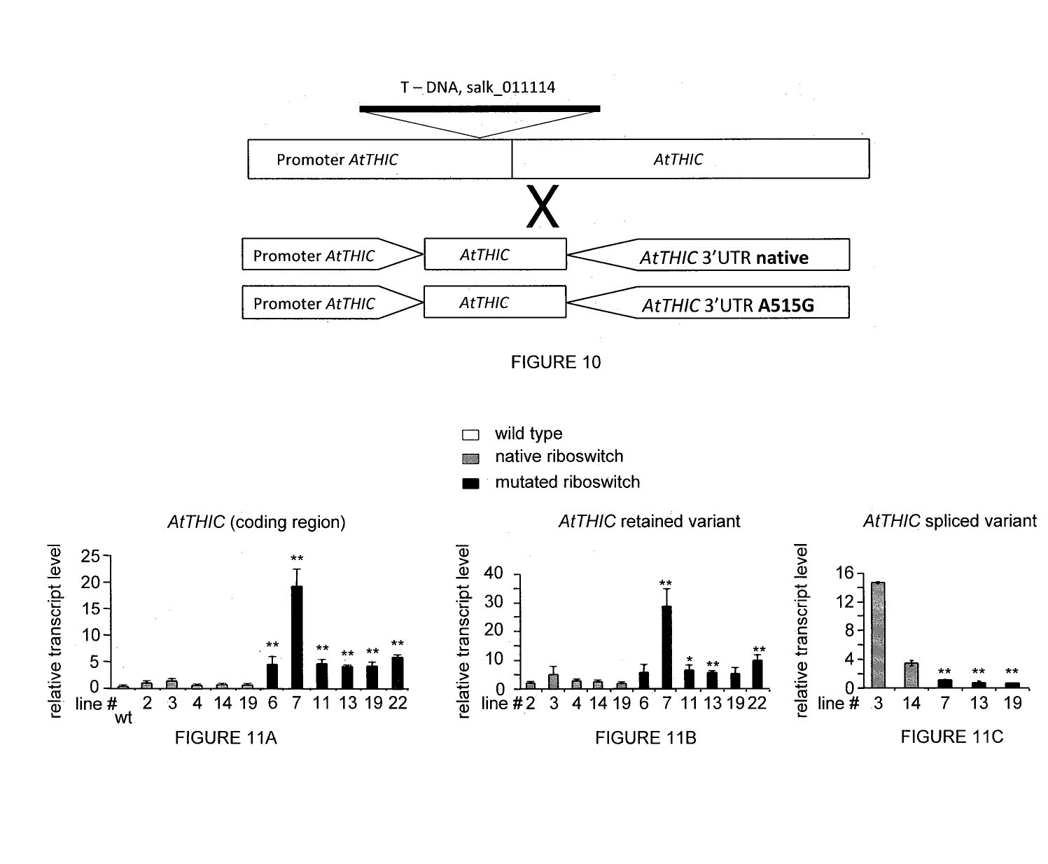

Arabidopsis T-DNA mutant plants, in which T-DNA insertion abolished AtTHIC expression (Kong D et al., 2008. ibid), were used to engineer transgenic plants with riboswitch deficiency. Two AtTHIC expression cassettes were generated, containing the promoter, gene, and the 3′ region of AtTHIC. In one of these cassettes, the AtTHIC 3′ region contained the native TPP riboswitch (this construct served as a control); the second cassette contained an A to G mutation (A515G, relative to the stop codon) in the TPP riboswitch, which renders it inactive (FIG. 10). These cassettes were introduced independently into the background of the Arabidopsis T-DNA mutants.

Monitoring the AtTHIC gene expression level revealed that its expression, as well as the expression of the intron-retained variant, were higher in the transgenic lines harboring a deficient TPP riboswitch compared to the expression in those carrying a functional one (FIG. 11A,B). The expression of the intron-spliced variant behaved oppositely (FIG. 11C). It should be noted that since the A515G mutation of the TPP riboswitch prevents intron splicing, the intron-spliced variant measured in the real-time RNA analysis could come from the poorly expressed endogenous AtTHIC gene, and not from the transgene. Given the constructs used in this study, it is not possible to design primers that could differentiate between the two.

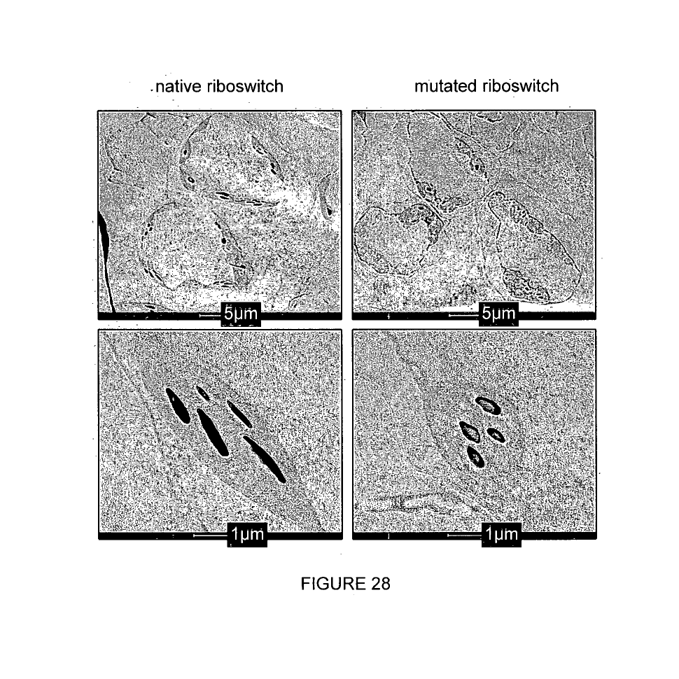

Interestingly, while plants harboring the native TPP riboswitch did not display any particular phenotype, those harboring the deficient TPP riboswitch exhibited chlorosis and growth retardation (observed in 10 independent lines of transformation; FIG. 27). Examination of the leaf chloroplast ultrastructure in these plants using transmission electron microscopy (TEM) revealed that chloroplasts derived from plants deficient in riboswitch activity were amorphous and possessed altered starch grain structure (FIG. 28).

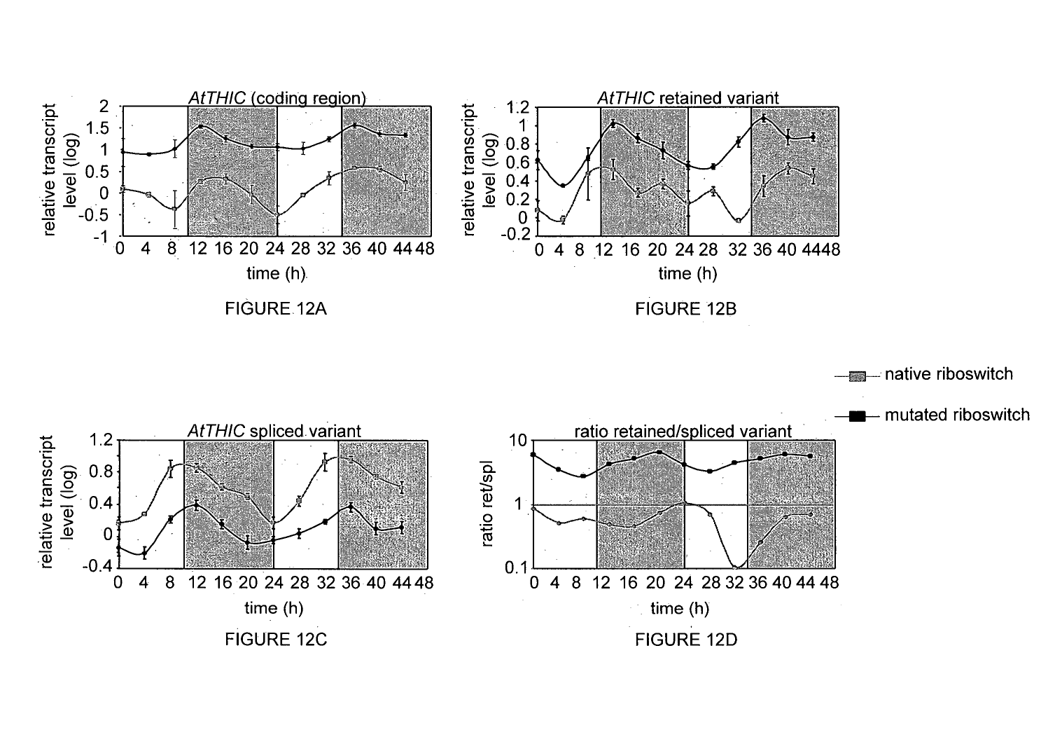

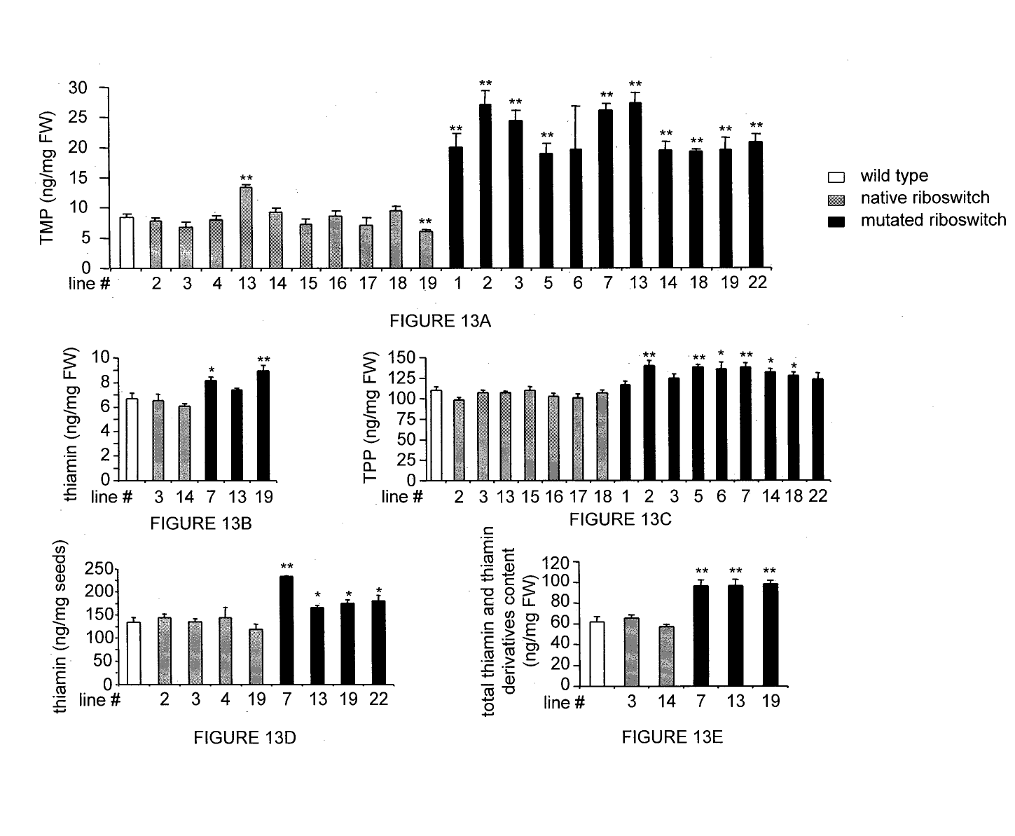

In a circadian assay on these transgenic plants it was found that AtTHIC circadian oscillations are not affected and that riboswitch deficiency causes the AtTHIC gene and its intron-retained variant to be up-regulated during the whole day period (FIG. 12 A, B), while the intron-spliced variant is down-regulated (FIG. 12C). Noticeably, in the transgenic plants harboring a deficient riboswitch, the ratio between the retained- and the spliced-variant shows that AtTHIC splicing favors the production of the retained variant throughout the day (FIG. 12D). This result indicates that TPP riboswitch deficiency elicited intron retention and thereby triggered the over-expression of AtTHIC. It was next evaluated if the levels of TMP, thiamine and TPP (synthesized natively in this order) have been altered due to riboswitch deficiency. The results showed that plants carrying a deficient riboswitch contained about three-fold increase in TMP levels as compared to the plants carrying a functional riboswitch and to wt plants (FIG. 13A), while thiamine (FIG. 13B) and TPP (FIG. 13C) contents were only moderately elevated. The levels of thiamine derivatives in seeds were also measured and it was found that riboswitch deficient plants accumulated ˜20% more thiamine in seeds, but did not accumulate TMP or TPP, which are normally absent in this sink tissue³⁴ (FIG. 13D). Nevertheless, the total amount of thiamine and its derivatives in the riboswitch deficiency plants was significantly higher compared to plants carrying a functional riboswitch and to wt plants (FIG. 13E).

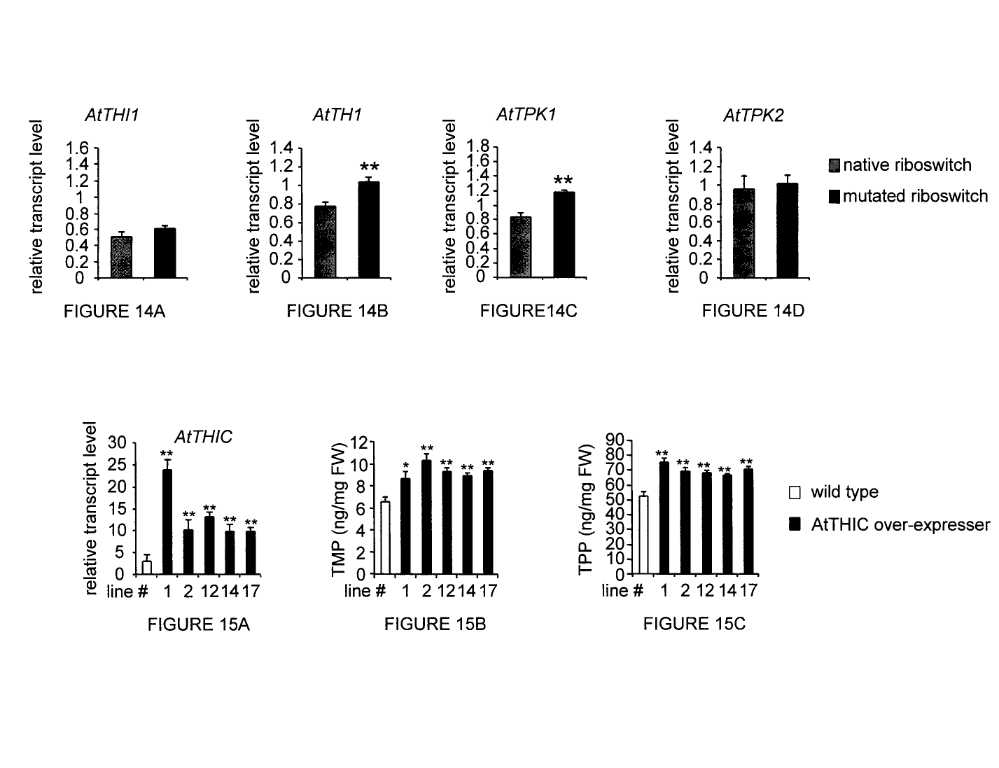

The expression levels of other thiamine biosynthetic genes was affected by riboswitch deficiency to a limited extend only (FIG. 14), suggesting that THIC overexpression was sufficient to increase TMP biosynthesis 3 folds. To further evaluate whether the phenotypes observed in the riboswitch deficient plants were a result of altered gene expression, an Arabidopsis whole-genome array was used to compare the transcriptome of plants with either functional or a deficient riboswitch. Following a false discovery rate (FDR) correction, this experiment showed that riboswitch deficiency did not cause significant changes at the transcriptional level. This confirmed that the reaction carried out by THIC may be the rate limiting step of TMP biosynthesis and that riboswitch deficiency directly increased thiamine biosynthesis at the post-transcriptional level.

To confirm that riboswitch deficiency increased TMP biosynthesis via AtTHIC overexpression, the AtTHIC coding sequence was expressed under the control of the AtUBIQUITINI promoter (Callis J et al., 1990. J Biol Chem 265, 12486-12493). In these plants, an elevation of AtTHIC expression, and an increase in TMP and TPP levels was observed (FIG. 15). In addition, these plants exhibited a chlorotic phenotype, which was observed in the independent line of transformation that displayed the highest AtTHIC expression level (line #1). These findings demonstrated that AtTHIC overexpression, whether it is caused by riboswitch deficiency or by altered promoter activity, increased TMP biosynthesis and may cause a chlorotic phenotype when AtTHIC is highly overexpressed. However, as the TPP riboswitch regulated pathway for thiamine synthesis is highly conserved in plants, bacteria, fungi and algae, modifying the riboswitch activity provide a universal means for producing elevated amounts of thiamine and/or thiamine derivatives.

### Example 4

### Effect of Riboswitch Deficiency on the Activity of Thiamine-Requiring Enzymes

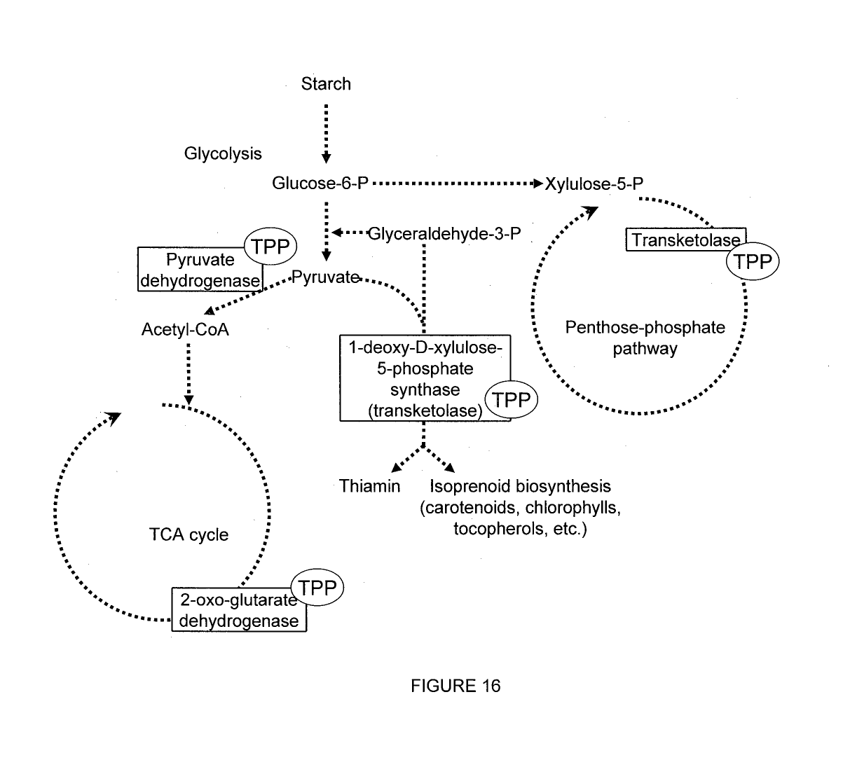

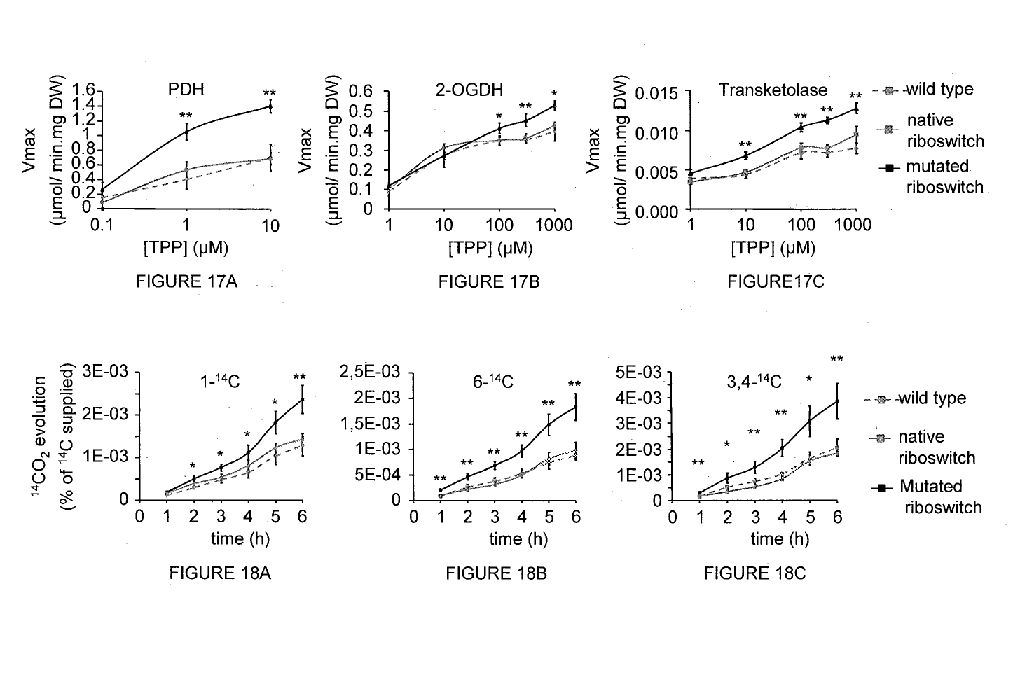

Thiamine monophosphate (TMP) and thiamine are the precursors for TPP biosynthesis, the later being an obligatory ligand for the key enzymes involved in both the TCA cycle and the pentose phosphate pathway (PPP; Frank R A et al., Cell Mol Life Sci 64, 892-905; FIG. 16). As exemplified herein above, TPP riboswitch disruption resulted in about three-fold increase in TMP but only in about 20% increase in TPP levels. To examine whether this difference is due to an enhanced TPP turnover by thiamine-dependent enzymes, enzymatic activities of three thiamine requiring enzymes (pyruvate dehydrogenase, PDH; 2-oxo-glutatarate dehydrogenase, 2-OGDH; and transketolase, TK) were measured in plants harboring the defective riboswitch. Plants harboring the construct with a native riboswitch and to wild type plants served as controls. Interestingly, thiamine requiring enzymes in plants deficient in riboswitch activity displayed higher enzymatic activities in the presence of increasing TPP concentrations as compared to the control and wt plants (FIG. 17). No effect was observed on the activity of five other enzymes involved in primary metabolism (AGPase; GAPDH; NAD-dependent ICDH; NADP-dependent ICDH; and Rubisco).

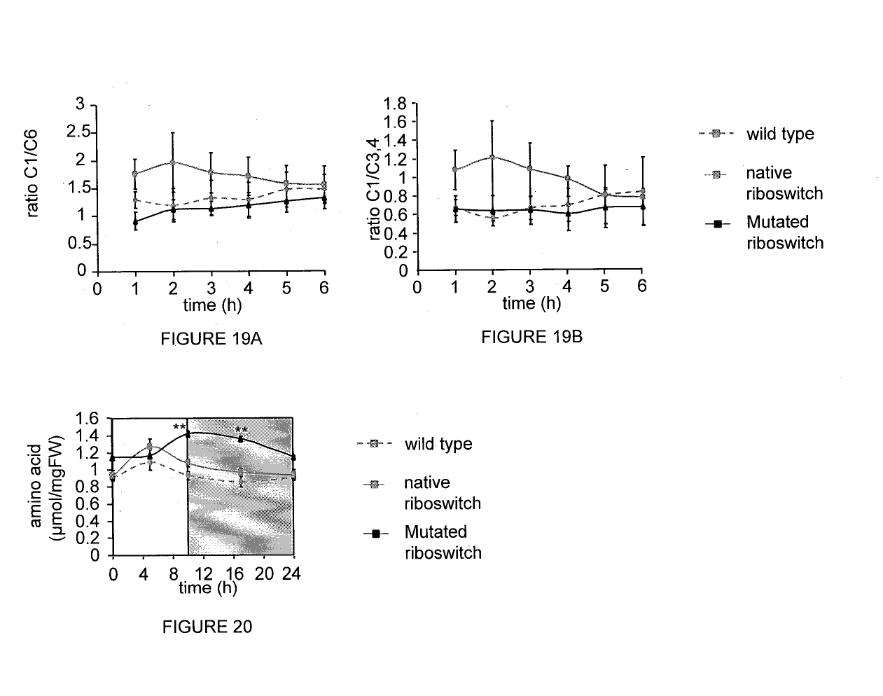

In addition, alteration in the metabolic fluxes through the TCA cycle and the PPP was examined in the plants deficient in riboswitch activity. For direct assessment of the defective riboswitch effect on the plant respiratory rate, the fluxes in the TCA and PPP pathways were estimated on the basis of ¹⁴CO₂ evolution. This was achieved by incubating leaf discs (isolated during the dark period) with [1-¹⁴C]-glucose, [3,4-¹⁴C]-glucose or [6-¹⁴C]-glucose over a period of 6 h. The consequent ¹⁴CO₂ emission was then measured at hourly intervals. The release of ¹⁴CO₂ from all positionally labeled glucoses were significantly higher in plants deficient in riboswitch activity as compared to the control and wt plants, which were very similar to each other (FIG. 18). However, the ratio of ¹⁴CO₂ evolution from the C1 or the C6 position of glucose to that from the C3,4 positions were similar in riboswitch deficient plants and in control plants (FIG. 19), providing evidence that riboswitch deficiency resulted in a general increase in respiration rate via both the TCA cycle and the PPP.

### Example 5

### Effect of Riboswitch Deficiency on Primary/Central Metabolism

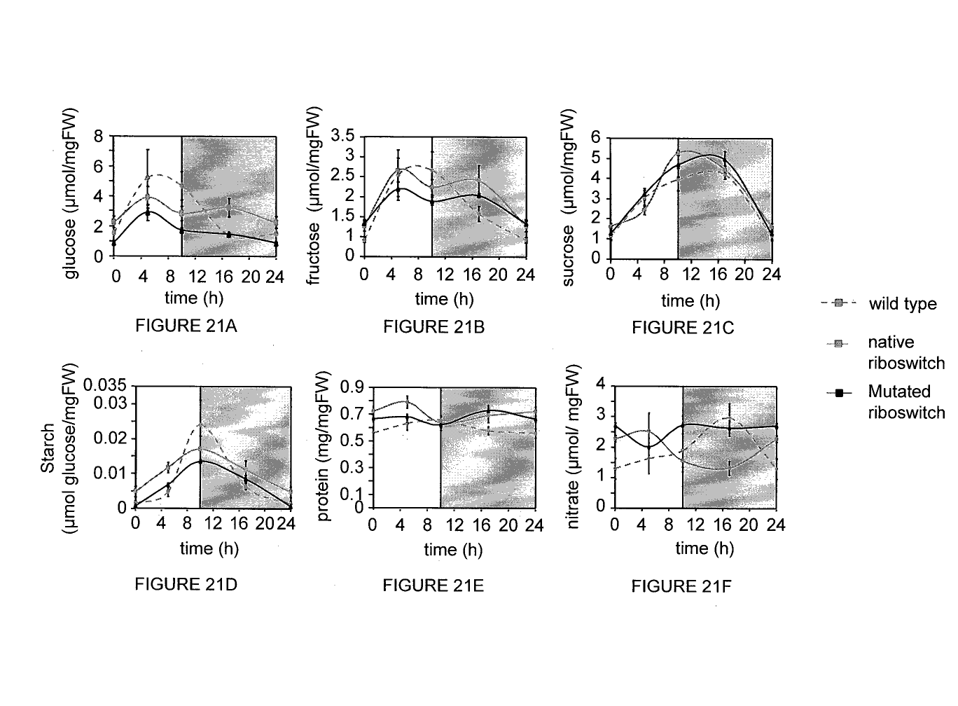

As exemplified hereinabove, riboswitch disruption resulted in increased flux through the TCA cycle and the PPP. Thus, it was further investigated to what extent these alterations affect the steady state levels of plant primary/central metabolites throughout the day. Four weeks old plants at four time points were harvested and the level of primary metabolites of interest was measured using colorimetric protocols (see Methods). Plants deficient in riboswitch activity displayed elevated total free amino acid content during the dark period only, compared to the control and wt plants, which were very similar throughout the day (FIG. 20). However, the level of glucose, fructose, sucrose, starch, protein, and nitrate remained practically unchanged (FIG. 21).

In order to further characterize the metabolic alterations that occurred in riboswitch deficient plants, an established gas chromatography mass spectrometry (GC-MS)-based metabolic profiling (Lisec J et al. 2006. Nat Protoc 1, 387-396) was performed during a diurnal period (4 time points, start and middle of both the light and dark periods). It appeared that the entire metabolic network was strongly affected. Notably, the steady state levels of 18 metabolites (out of 43 identified) differed significantly in the plants harboring a deficient riboswitch in at least one time point. Significant changes included accumulation of six amino acids (alanine, β-alanine, aspartate, threonine, proline, tryptophan) and reduction of seven other amino acids (GABA, glycine, methionine, histidine, glutamate, tyrosine, phenylalanine). The increased flux through the TCA cycle also generated a higher steady state level of amino acid precursors such as isocitrate during the dark period, and a lower steady state level of precursors such as succinate during the entire day period. Interestingly, the steady state levels of glutamine and GABA were decreased. Without wishing to be bound by any theory or mechanism of action, the decrease may be attributed to the fact that these two compounds serve as alternative carbon donors for the TCA cycle. In addition, a significant increase in spermine and tyramine level was observed, suggesting an augmentation of both β-alanine and hydroxycinnamic acid-tyramine amide biosynthetic pathways.

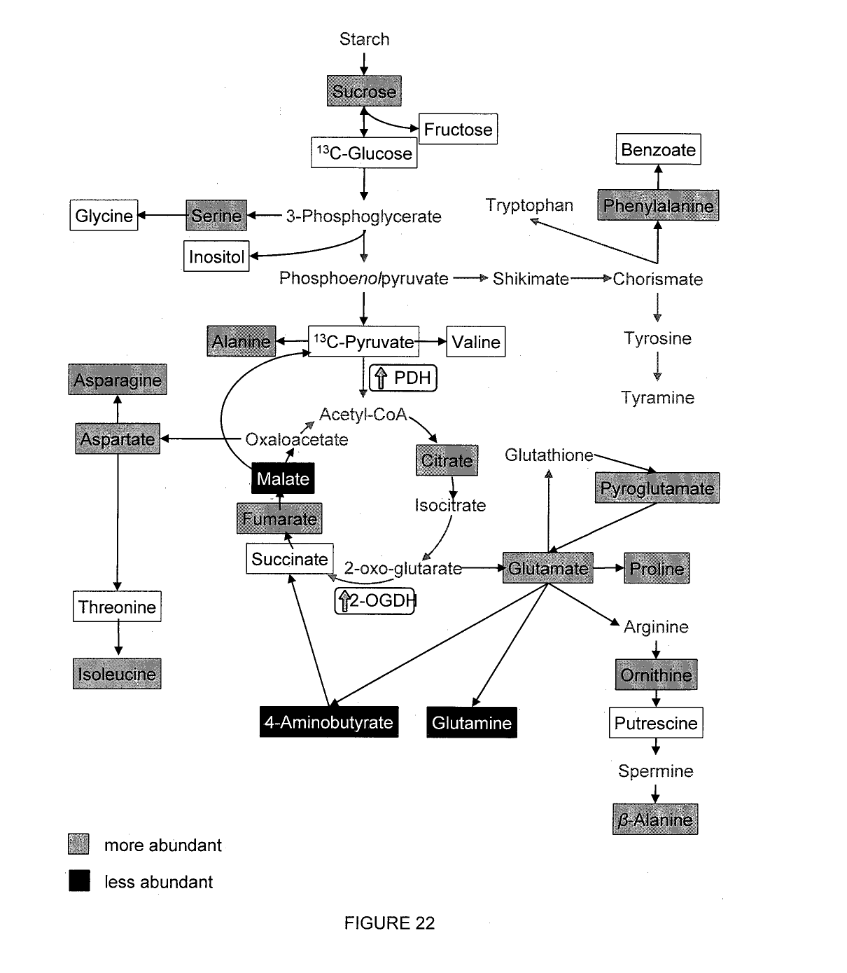

To further characterize how riboswitch malfunction affects the fluxes through the TCA cycle, isotope labeling experiments were performed. The relative isotope redistribution in leaf discs fed with [U-¹³C]-glucose or [U-¹³C]-pyruvate was evaluated and further processed using a GC-MS approach that facilitates isotope tracing (Roessner-Tunali U et al., 2004. Plant J 39, 668-679). Interestingly, an increase in label redistribution to most amino acids was observed (aspartate, asparagine, isoleucine, alanine, β-alanine, proline, phenylalanine, serine; FIG. 22). Additionally, an augmentation in label redistribution was detected in the TCA cycle intermediates citrate and fumarate as well as glutamate. In contrast, a reduction in label redistribution was observed for malate, glutamine and GABA, metabolites.

### Example 6

### Effect of Riboswitch Deficiency on Isoprenoid Metabolism, Photosynthetic Activity and Specialized Metabolism

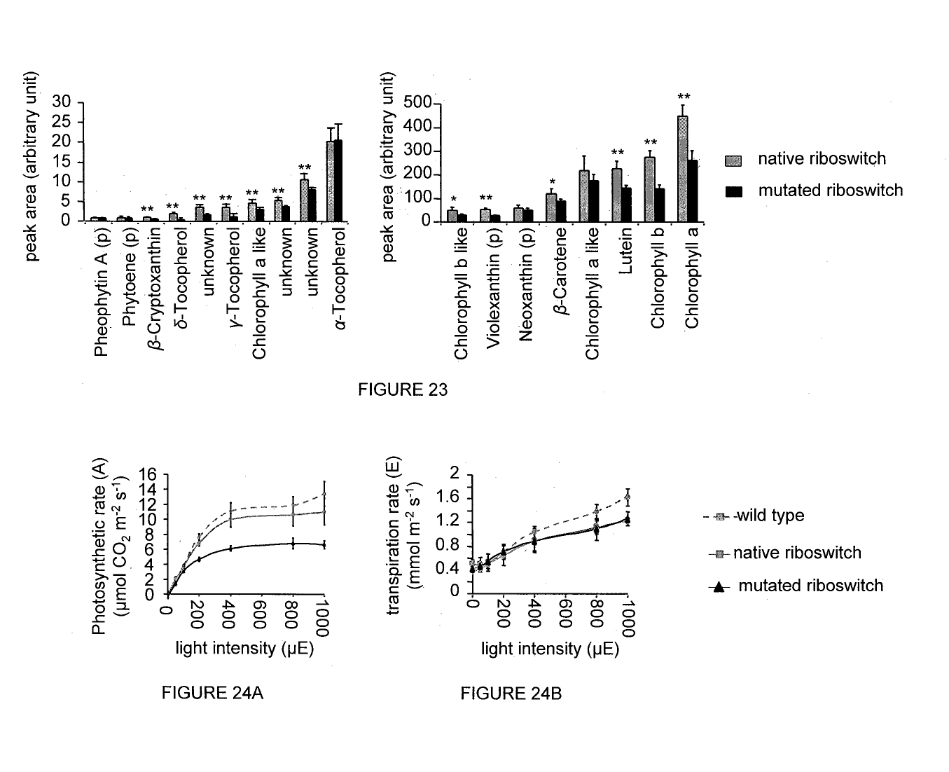

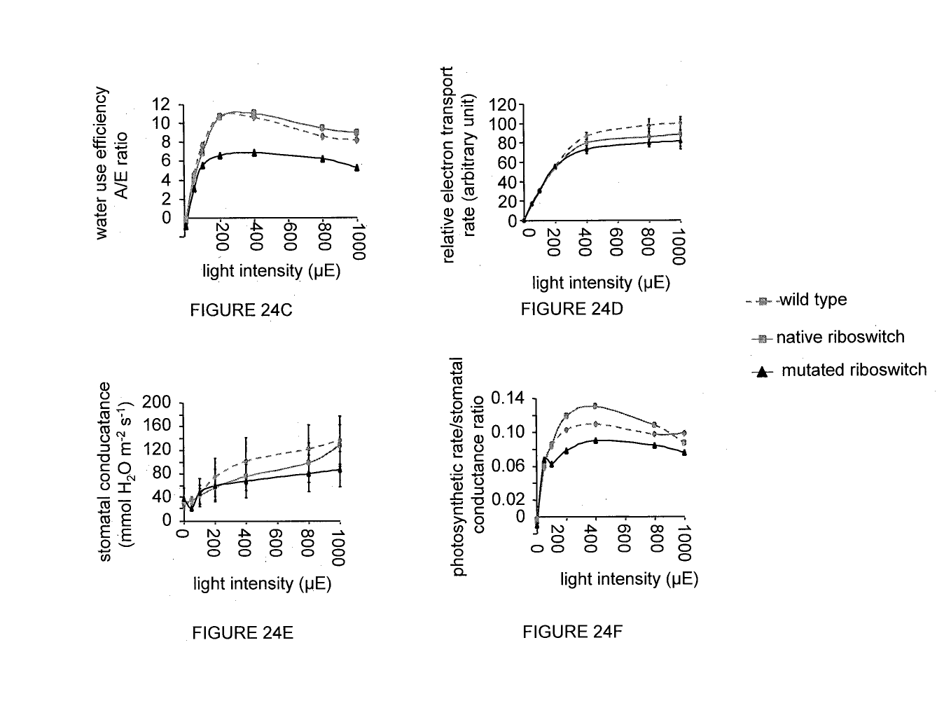

As exemplified herein, plants deficient in TPP riboswitch activity displayed phenotypes of chlorosis. Metabolic profiling of compounds belonging to the isoprenoids pathway was thus performed, using an established HPLC-PDA-based protocol (Fraser P. 2000; ibid). Riboswitch-deficient plants accumulated significantly less isoprenoids including chlorophyll a and b, δ- and γ-tocopherol, β-cryptoxanthin, violexanthin and neoxanthin as compared to wt and control plants (FIG. 23). The lower chlorophyll content observed in these plants led us to investigate whether these plants also exhibited altered photosynthetic rates. Gas exchange was subsequently analyzed in vivo in 10 week-old plants under photon flux densities that ranged from 0 to 1000 μmol m⁻² s⁻¹. The results indicated a reduced photosynthetic rate of the plants harboring a defective riboswitch compared to that of wt and control plants, while stomatal conductance, electron transfer rate (ETR), and transpiration rate were unaffected (FIG. 24). Without wishing to be bound by any specific theory or mechanism of action, it is suggested that the reduction in photosynthesis in the model plant Arabidopsis was a consequence of the lower chlorophyll content of the plants rather than a direct effect on the photosynthetic machinery.

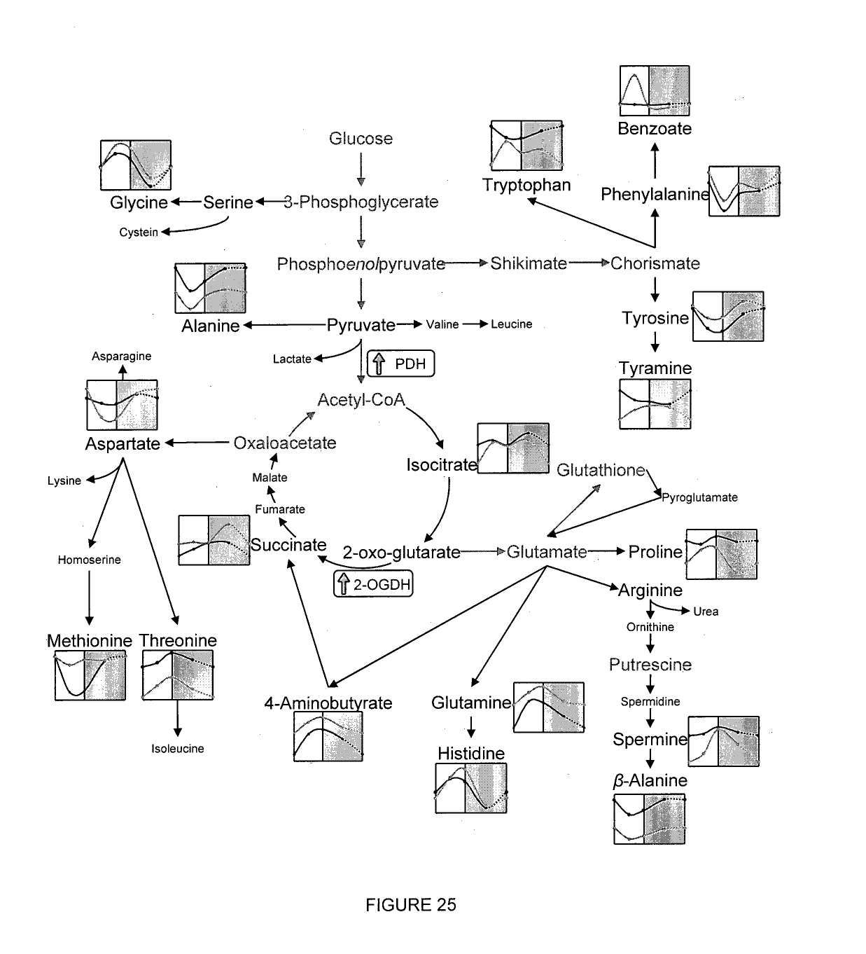

To evaluate the broader consequences of altering riboswitch activity in plants metabolomic analysis was performed using liquid chromatography mass-spectrometry (LC-MS, Malitsky S et al., 2000; ibid). This system allows the detection of mainly semi polar, specialized (i.e. secondary) metabolites. Comparing the metabolite profiles of plants harboring a defective riboswitch to those harboring a functional one and wt, revealed that riboswitch deficiency dramatically altered secondary metabolism. We also observed larger abundance of differential mass signals in the middle and in the end of the dark photoperiod, in accordance with our previous finding that the metabolic phenotype caused by riboswitch deficiency was more pronounced during this period (FIG. 25).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A thiamine producing bioengineered organism comprising a modified thiamine pyrophosphate (TPP)-responsive riboswitch having reduced affinity to TPP, wherein the organism produces elevated amounts of thiamine and/or derivatives thereof compared to a corresponding organism comprising an unmodified TPP-responsive riboswitch.

2. The organism of claim 1, said organism is selected from the group consisting of bacteria, fungi, algae and plants.

3. (canceled)

4. (canceled)

5. The organism of claim 1, wherein the TPP-responsive riboswitch is located within an untranslated sequence of a thiamine synthase gene.

6. The organism of claim 5, wherein the thiamine synthase gene is selected from the group consisting of the endogenous thiamine synthase gene of the organism and an exogenous gene.

7. The organism of claim 6, said organism comprises an expression cassette comprising a promoter sequence, a polynucleotide encoding thiamine synthase and an untranslated sequence comprising a modified riboswitch having reduced affinity to TPP.

8. The organism of claim 7, wherein the untranslated sequence comprising the modified riboswitch is located at a position selected from the group consisting of upstream (5′) to the thiamine synthase coding region, downstream (3′) to the thiamine synthase coding region and within the thiamine synthase coding region.

9. (canceled)

10. (canceled)

11. The organism of claim 7, wherein the promoter is selected from the group consisting of said organism's native thiamine synthase promoter and a heterologous promoter.

12. (canceled)

13. (canceled)

14. The organism of claim 7, wherein the encoded thiamine synthase is an Arabidopsis thiamine C synthase (AtTHIC).

15. The organism of claim 14, wherein the untranslated sequence comprises a point mutation.

16. The organism of claim 15, wherein the point mutation is a substitution of A to G at position 515 (A515G) relative to the stop codon of the Arabidopsis thiamine C synthase gene (AtTHIC).

17. The organism of claim 16, wherein the expression cassette comprises a polynucleotide having the nucleic acid sequence set forth in SEQ ID NO:3.

18. The organism of claim 1, said organism is further modified to have reduced activity of thiamine pyrophosphate producing enzyme.

19. The organism of claim 18, wherein the thiamine pyrophosphate producing enzyme is selected from the group consisting of thiamine phosphate kinase (TPhK) and thiamine pyrophosphokinase (TPyK).

20. The organism of claim 19, wherein the thiamine phosphate kinase (TPhK) is encoded by a polynucleotide having the nucleic acid sequence set forth in SEQ ID NO:6, and the thiamine pyrophosphokinase (TPyK) is encoded by a polynucleotide having the nucleic acid sequence set forth in any one of SEQ ID NOS:8, 10, 12, 14, 16 and 18.

21. The organism of claim 20, wherein the thiamine phosphate kinase comprises the amino acids sequence set forth in SEQ ID NO:5 and the thiamine pyrophosphokinase comprises the amino acids sequence set forth in any one of SEQ ID NOS:7, 9, 11, 13, 15 and 17.

22. (canceled)

23. (canceled)

24. A method for producing elevated amounts of thiamine and derivatives thereof by a thiamine-producing organism, the method comprising inserting at least one modification within a thiamine pyrophosphate (TPP)-responsive riboswitch polynucleotide sequence, wherein the modification results in reduced affinity of the riboswitch to TPP, thereby obtaining an organism producing elevated amounts of thiamine and/or derivatives thereof compared to a corresponding wild type organism.

25. (canceled)

26. The method of claim 24, wherein the TPP-responsive riboswitch is located within an untranslated sequence of a thiamine synthase gene.

27. The method of claim 24, further comprising reducing the expression or activity of a thiamine pyrophosphate producing enzyme.

28. The method of claim 24, wherein inserting the modification comprises introducing a mutation in the TPP-responsive riboswitch polynucleotide sequence.

29. A method for producing elevated amounts of thiamine and derivatives thereof by a thiamine-producing organism, the method comprising transforming at least one organism cell with an expression cassette comprising a promoter, a polynucleotide encoding thiamine synthase and an untranslated sequence comprising modified riboswitch having reduced affinity to thiamine pyrophosphate (TPP), thereby obtaining an organism producing elevated amounts of thiamine and derivatives thereof compared to a corresponding wild type organism.

30. The method of claim 29, wherein the untranslated sequence comprising the modified riboswitch is located at a position selected from the group consisting of upstream (5′) to the thiamine synthase coding region, downstream (3′) to the thiamine synthase coding region and within the thiamine synthase coding region.

31-34. (canceled)

35. The method of claim 29, wherein the thiamine synthase is Arabidopsis thiamine C synthase (AtTHIC).

36. The method of claim 35, wherein the modified riboswitch comprises a point mutation.

37. The method of claim 36, wherein the point mutation is a substitution of A to G at position 515 (A515G) relative to the stop codon of an Arabidopsis thiamine C synthase gene (AtTHIC).

38. The method of claim 37 wherein the expression cassette comprises a polynucleotide having the nucleic acids sequence set forth in SEQ ID NO:3.

39. (canceled)

40. The organism of claim 1, wherein the TPP-responsive modified riboswitch comprises a point mutation.


SEQUENCE LISTING (45 sequences)

SEQ ID NO: 1 - protein, 644 aa, Arabidopsis thaliana
MAASVHCTLMSVVCNNKNHSARPKLPNSSLLPGFDVVVQAAATRFKKETTTTRATLTFDPPTTNSERAKQRKHTIDPSSPDFQPIPSFEECFPKSTKEHKEVVHEESGHVLKVPFRRVHLSGGEPAFDNYDTSGPQNVNAHIGLAKLRKEWIDRREKLGTPRYTQMYYAKQGIITEEMLYCATREKLDPEFVRSEVARGRAIIPSNKKHLELEPMIVGRKFLVKVNANIGNSAVASSIEEEVYKVQWATMWGADTIMDLSTGRHIHETREWILRNSAVPVGTVPIYQALEKVDGIAENLNWEVFRETLIEQAEQGVDYFTIHAGVLLRYIPLTAKRLTGIVSRGGSIHAKWCLAYHKENFAYEHWDDILDICNQYDVALSIGDGLRPGSIYDANDTAQFAELLTQGELTRRAWEKDVQVMNEGPGHVPMHKIPENMQKQLEWCNEAPFYTLGPLTTDIAPGYDHITSAIGAANIGALGTALLCYVTPKEHLGLPNRDDVKAGVIAYKIAAHAADLAKQHPHAQAWDDALSKARFEFRWMDQFALSLDPMTAMSFHDETLPADGAKVAHFCSMCGPKFCSMKITEDIRKYAEENGYGSAEEAIRQGMDAMSEEFNIAKKTISGEQHGEVGGEIYLPESYVKAAQK

SEQ ID NO: 2 - DNA, 2435 bp, Arabidopsis thaliana
gactcactcagtgtgcgcgattcatttcaaaaacgagccagcctcttcttccttcgtctactagatcagatccaaagcttcctcttccagctatggctgcttcagtacactgtaccttgatgtccgtcgtatgcaacaacaagaatcactctgctcggccgaaacttccaaactcgtctttgttacctggattcgatgttgttgttcaagctgctgctactcgattcaagaaggaaacaacaaccacaagagccactttgacgtttgatccaccaaccactaattctgagagagctaagcagagaaaacacaccattgatccttcttctcctgattttcaaccaattccatcttttgaagaatgctttcctaagagcactaaagaacacaaggaagttgtccatgaagaatctggtcatgttcttaaagttccctttcgtcgtgttcatttgtctggtggtgagccagcttttgataattatgacactagtggtcctcaaaatgttaatgcacacattgggcttgctaagctaaggaaggagtggattgatcgtcgggagaagctaggaacaccaagatacactcaaatgtactacgctaagcaagggatcataactgaggaaatgctctactgtgctactagggagaagctagaccctgagtttgtaagatcagaagttgcacgaggacgggcgattatcccttccaacaagaagcatttggagctggaaccgatgattgttggtagaaagttcttggtcaaggtcaatgcgaatatcggaaactctgctgttgccagctctattgaagaggaagtctacaaggttcagtgggcaaccatgtggggagctgatacaatcatggatctctcaactggtcgtcacatccatgagacacgtgagtggatcctaaggaattcagctgtgcctgttggtacggtgcctatttatcaagcacttgagaaagtggatggaattgctgagaatcttaactgggaggttttcagagagactctgattgaacaagctgagcaaggtgtagactatttcacaatccatgctggagttttgctgcgttacattcccttaactgccaagcgtttgaccgggatcgtttcgcgtggaggatccattcatgctaaatggtgcttagcttaccacaaggagaactttgcttacgagcactgggatgacattctagacatctgtaaccagtatgatgtggctctttccattggagatggtctgagacctggctccatttatgatgctaacgacactgctcagtttgcagagctccttactcaaggtgaactaactcgccgagcgtgggaaaaagatgtgcaggtgatgaatgaagggccagggcatgtcccaatgcacaagattccagagaatatgcagaagcagttggagtggtgtaacgaggcaccattctacacccttggtcctttgactactgatattgcccctggatatgatcacattacctctgccattggagctgccaatattggagccttgggtacagctcttctttgctatgtaacaccaaaagaacaccttgggctaccaaacagggacgatgtgaaggccggggttatagcatacaagatcgccgctcatgcagctgatctagccaaacagcatccacatgctcaggcatgggacgatgcgctgagcaaagcgcggtttgagtttagatggatggaccaatttgctctgtcgttggaccccatgactgctatgtctttccatgatgaaactcttccagctgatggagccaaggttgcacacttttgctccatgtgtggaccaaaattctgctctatgaagataacagaagacatccgaaagtatgcagaggagaatggttatggcagtgctgaagaagcaatcagacaaggaatggatgctatgagtgaagaattcaacatcgcaaagaaaaccattagcggagaacagcacggtgaagtaggtggagaaatatatttgccagagagctatgtcaaagctgctcagaaataaaaggcaaatgttttaaacaagaccttgcttacccaagtcttggtgcctgttggactatacctggataaaggcacaaactgttggggtgcttgaaccaggatagcctgcgaaaaggcgggctatccgggaccaggctgagaaagtccctttgaacctgaacagggtaatgcctgcgcagggagtgtgcagttttttttttttcctgtagctttctaaaggagaagaagctactgttgccgctcgagtctcgttccacggttttcaacagttagtttcttatgagctaagagattcagcttaattggcttacagccataaaagaagtctttaactgatgcactaagtcactaacagtagggaataattcaatcaaaaaatcatccagattgataaaaatgcatttgcacc

SEQ ID NO: 3 - DNA, 5173 bp, Artificial Sequence
  Expression cassette comprising the mutated riboswitch
tttcttctccttctagtgaatcaaacaaacctgaaacataaaaatcattataagacctaaaacacacacgaaatgatcaaaggtattgaaggaaaacttcaaaattgatgaagaagagaggtcaagcttacctttgaaatgattaccagtcaaaatagttttggaggggttttaattgcagatttagaaaaaaatttctctgttttcttaaaaacatgatgtcggtgtggtagagaggggatggttttatgtacggatcggatcgtgcggggaagacaaaatagaaaaacaacgagggagttagttgcttacatgttgtttttcaaagatattattttcttcttattacatacacttttgaatttgttgatcgtgttacttacataaaattgcaggttaggtccctttgttttcgcagtttttgcaattatttctcatatttcttaatattgggcttttcacatgtaataagcccaacgataagaccatgacaatttctatacgaaacatgatataaattctttggatatacattatgaatttacgatatacaattagtttgtttaaatatcaaaatataaatgcgtcaatggttgttgttacttgtgagattatctttctatttaagaagaataattctcttcgtagataaatttttaaaataattttccgagttttctaatgtttctagatatgatttgatttgaacaattaattcgtggttctttgaatgaatatatcgactgtatttgatttcagttaaactgataataattgtcatttacgtctcaaaagaattgaaatatcatgtctctcaagatatggacttacatattgttatgcattatttatcaaaatatgtggacaaaacataatatcaatgtcgctttcagaataattgaacaacagatattgagaaatcaatttttatggttatatcaattgtcattgccaacatctattacatagtaacagtccaatttacattacaatggtaattcaatgaaggtaatttactttttattggtttactcgtgaaacgacgttctcctcctcacgtaccttatcttaatatcctgatcaacggacaccaattttcgacaaaatatctgagaaagaggacacgtcagcaagcctttcgctttaggctgcattgggccgtgacaatattcagacgattcaggaggttcgttccttttttaaaggaccctaatcactctgagtaccactgactcactcagtgtgcgcgattcatttcaaaaacgagccagcctcttcttccttcgtctactagatcagatccaaagcttcctcttccaggttcgaatccttgatttctccatgaatgtgcatggtagtccaacaattgtcgatgtttttgatagagagttttgtagattttctccggcgaaattccgatttgttcttcaatattatgtgcatgaaactttttttttaagattgtgcgtttagatgcaatattcgactctttgttgttctcatgctcgtcgatttcgatgtgtttctgttaatccattgatcgtatcggaaactgtgattgattgattcatattttcgtttgtctccagctatggctgcttcagtacactgtaccttgatgtccgtcgtatgcaacaacaagaatcactctgctcggccgaaacttccaaactcgtctttgttacctggattcgatgttgttgttcaagctgctgctactcgattcaagaaggaaacaacaaccacaagagccactttgacgtttgatccaccaaccactaattctgagagagctaagcagagaaaacacaccattgatccttcttctcctgattttcaaccaattccatcttttgaagaatgctttcctaagagcactaaagaacacaagtaattgcttcacttaatctacattttttcatattggaagagttgagaaatcactggttggtttttggttgttttcagggaagttgtccatgaagaatctggtcatgttcttaaagttccctttcgtcgtgttcatttgtctggtggtgagccagcttttgataattatgacactagtggtcctcaaaatgttaatgcacacattggtatgtgattccacctcgtgtttactttacacattcacctctcttttatgtgactatcgataaatgaaacttaccaagcagggcttgctaagctaaggaaggagtggattgatcgtcgggagaagctaggaacaccaagatacactcaaatgtactacgctaagcaagggatcataactgaggaaatgctctactgtgctactagggagaagctagaccctgagtttgtaagatcagaagttgcacgaggacgggcgattatcccttccaacaagaagcatttggagctggaaccgatgattgttggtagaaagttcttggtcaaggtcaatgcgaatatcggaaactctgctgttgccagctctattgaagaggaagtctacaaggttcagtgggcaaccatgtggggagctgatacaatcatggatctctcaactggtcgtcacatccatgagacacgtgagtggatcctaaggaattcagctgtgcctgttggtacggtgcctatttatcaagcacttgagaaagtggatggaattgctgagaatcttaactgggaggttttcagagagactctgattgaacaagctgagcaaggtgtagactatttcacaatccatgctggagttttgctgcgttacattcccttaactgccaagcgtttgaccgggatcgtttcgcgtggaggatccattcatgctaaatggtgcttagcttaccacaaggagaactttgcttacgagcactgggatgacattctagacatctgtaaccagtatgatgtggctctttccattggagatggtctgagacctggctccatttatgatgctaacgacactgctcagtttgcagagctccttactcaaggtgaactaactcgccgagcgtgggaaaaagatgtgcaggtatactacaactacttatctaattcacttatattcatccagtttgtctttggatacaactacttatatctacttttccaggtgatgaatgaagggccagggcatgtcccaatgcacaagattccagagaatatgcagaagcagttggagtggtgtaacgaggcaccattctacacccttggtcctttgactactgatattgcccctggatatgatcacattacctctgccattggagctgccaatattggagccttgggtacagctcttctttgctatgtaacaccaaaagaacaccttgggctaccaaacagggacgatgtgaaggccggggttatagcatacaagatcgccgctcatgcagctgatctagccaaacagcatccacatgctcaggcatgggacgatgcgctgagcaaagcgcggtttgagtttagatggatggaccaatttgctctgtcgttggaccccatgactgctatgtctttccatgatgaaactcttccagctgatggagccaaggttgcacacttttgctccatgtgtggaccaaaattctgctctatgaagataacagaagacatccgaaagtatgcagaggagaatggttatggcagtgctgaagaagcaatcagacaaggaatggatgctatgagtgaagaattcaacatcgcaaagaaaaccattagcggagaacagcacggtgaagtaggtggagaaatatatttgccagagagctatgtcaaagctgctcagaaataaaaggtcagtatgtttagactgttagtcgttgctttctcaacaaacatgttagttactgcatgctagtataaaatcattcaggtttataatcttttcttaaatctgcaacatatggtcaactcttaaatgagtccttactgtgatctttgttttttatcgtgtttctttttcttctgctgcatcaggcaaatgttttaaacaagaccttgcttacccaagtcttggtgcctgttggactatacctggataaaggcacaaactgttggtaagcttagtagtctctatgtcatgttacttttagaactatctatgttgtctgttcatttgagtcagagtcagcaataaagacaatctaagttgatgtttcaatacttttttgtgtgatttggttggtgaattgacatgcaaaagcaccaggggtgcttgaaccaggatagcctgcgaaaaggcgggctatccgggaccaggctgagaaagtccctttgaacctgaacagggtaatgcctgcgcagggggtgtgcagttttttttttttcctgtagctttctaaaggagaagaagctactgttgccgctcgagtctcgttccacggttttcaacagttagtttcttatgagctaagagattcagcttaattggcttacagccataaaagaagtctttaactgatgcactaagtcactaacagtagggaataattcaatcaaaaaatcatccagattgataaaaatgcatttgcacctttggggcataagctgaaattactctgctcgcacaaatcagatttttaaaacttatgatcatgttttcaacttatactcgtttgtttacataatgggatgatcagttgttcactttgagcaaagcatactgggcaggtgtagggaaagtgaagctcatgaaactgatccactgtaattctcattgttcagcttttatttacacaaatgtgcgagtcaacacgtaaaagattatgatatattgcagtagaacataagtaaaacgaccttaccaaatgcgaaagaaagcttttcgagccggaatgatcaaaaggttacacattctgtactcattttcctgctatatgagatcacacaattatcaagagcctgaaactctggaataccaatatttagacacgttacatagagttttgaaggtttctcatttgaccagctcctgtgtttgacatgctttggtttcatatggttaatgtttctgctaaatcaaccatgagagatgtgaactatttctatatcatgtgtctgtaacgttggggccgcttgtgagcctcttccttcaccattttcagtttgatcagttaataacctcatgtaaaagcagtgagagagagaagagatcagagatggcacactgtttcctttggaaatcctgggcgttgttatg

SEQ ID NO: 4 - DNA, 5173 bp, Artificial Sequence
  Expression cassette comprising the wild type riboswitch
tttcttctccttctagtgaatcaaacaaacctgaaacataaaaatcattataagacctaaaacacacacgaaatgatcaaaggtattgaaggaaaacttcaaaattgatgaagaagagaggtcaagcttacctttgaaatgattaccagtcaaaatagttttggaggggttttaattgcagatttagaaaaaaatttctctgttttcttaaaaacatgatgtcggtgtggtagagaggggatggttttatgtacggatcggatcgtgcggggaagacaaaatagaaaaacaacgagggagttagttgcttacatgttgtttttcaaagatattattttcttcttattacatacacttttgaatttgttgatcgtgttacttacataaaattgcaggttaggtccctttgttttcgcagtttttgcaattatttctcatatttcttaatattgggcttttcacatgtaataagcccaacgataagaccatgacaatttctatacgaaacatgatataaattctttggatatacattatgaatttacgatatacaattagtttgtttaaatatcaaaatataaatgcgtcaatggttgttgttacttgtgagattatctttctatttaagaagaataattctcttcgtagataaatttttaaaataattttccgagttttctaatgtttctagatatgatttgatttgaacaattaattcgtggttctttgaatgaatatatcgactgtatttgatttcagttaaactgataataattgtcatttacgtctcaaaagaattgaaatatcatgtctctcaagatatggacttacatattgttatgcattatttatcaaaatatgtggacaaaacataatatcaatgtcgctttcagaataattgaacaacagatattgagaaatcaatttttatggttatatcaattgtcattgccaacatctattacatagtaacagtccaatttacattacaatggtaattcaatgaaggtaatttactttttattggtttactcgtgaaacgacgttctcctcctcacgtaccttatcttaatatcctgatcaacggacaccaattttcgacaaaatatctgagaaagaggacacgtcagcaagcctttcgctttaggctgcattgggccgtgacaatattcagacgattcaggaggttcgttccttttttaaaggaccctaatcactctgagtaccactgactcactcagtgtgcgcgattcatttcaaaaacgagccagcctcttcttccttcgtctactagatcagatccaaagcttcctcttccaggttcgaatccttgatttctccatgaatgtgcatggtagtccaacaattgtcgatgtttttgatagagagttttgtagattttctccggcgaaattccgatttgttcttcaatattatgtgcatgaaactttttttttaagattgtgcgtttagatgcaatattcgactctttgttgttctcatgctcgtcgatttcgatgtgtttctgttaatccattgatcgtatcggaaactgtgattgattgattcatattttcgtttgtctccagctatggctgcttcagtacactgtaccttgatgtccgtcgtatgcaacaacaagaatcactctgctcggccgaaacttccaaactcgtctttgttacctggattcgatgttgttgttcaagctgctgctactcgattcaagaaggaaacaacaaccacaagagccactttgacgtttgatccaccaaccactaattctgagagagctaagcagagaaaacacaccattgatccttcttctcctgattttcaaccaattccatcttttgaagaatgctttcctaagagcactaaagaacacaagtaattgcttcacttaatctacattttttcatattggaagagttgagaaatcactggttggtttttggttgttttcagggaagttgtccatgaagaatctggtcatgttcttaaagttccctttcgtcgtgttcatttgtctggtggtgagccagcttttgataattatgacactagtggtcctcaaaatgttaatgcacacattggtatgtgattccacctcgtgtttactttacacattcacctctcttttatgtgactatcgataaatgaaacttaccaagcagggcttgctaagctaaggaaggagtggattgatcgtcgggagaagctaggaacaccaagatacactcaaatgtactacgctaagcaagggatcataactgaggaaatgctctactgtgctactagggagaagctagaccctgagtttgtaagatcagaagttgcacgaggacgggcgattatcccttccaacaagaagcatttggagctggaaccgatgattgttggtagaaagttcttggtcaaggtcaatgcgaatatcggaaactctgctgttgccagctctattgaagaggaagtctacaaggttcagtgggcaaccatgtggggagctgatacaatcatggatctctcaactggtcgtcacatccatgagacacgtgagtggatcctaaggaattcagctgtgcctgttggtacggtgcctatttatcaagcacttgagaaagtggatggaattgctgagaatcttaactgggaggttttcagagagactctgattgaacaagctgagcaaggtgtagactatttcacaatccatgctggagttttgctgcgttacattcccttaactgccaagcgtttgaccgggatcgtttcgcgtggaggatccattcatgctaaatggtgcttagcttaccacaaggagaactttgcttacgagcactgggatgacattctagacatctgtaaccagtatgatgtggctctttccattggagatggtctgagacctggctccatttatgatgctaacgacactgctcagtttgcagagctccttactcaaggtgaactaactcgccgagcgtgggaaaaagatgtgcaggtatactacaactacttatctaattcacttatattcatccagtttgtctttggatacaactacttatatctacttttccaggtgatgaatgaagggccagggcatgtcccaatgcacaagattccagagaatatgcagaagcagttggagtggtgtaacgaggcaccattctacacccttggtcctttgactactgatattgcccctggatatgatcacattacctctgccattggagctgccaatattggagccttgggtacagctcttctttgctatgtaacaccaaaagaacaccttgggctaccaaacagggacgatgtgaaggccggggttatagcatacaagatcgccgctcatgcagctgatctagccaaacagcatccacatgctcaggcatgggacgatgcgctgagcaaagcgcggtttgagtttagatggatggaccaatttgctctgtcgttggaccccatgactgctatgtctttccatgatgaaactcttccagctgatggagccaaggttgcacacttttgctccatgtgtggaccaaaattctgctctatgaagataacagaagacatccgaaagtatgcagaggagaatggttatggcagtgctgaagaagcaatcagacaaggaatggatgctatgagtgaagaattcaacatcgcaaagaaaaccattagcggagaacagcacggtgaagtaggtggagaaatatatttgccagagagctatgtcaaagctgctcagaaataaaaggtcagtatgtttagactgttagtcgttgctttctcaacaaacatgttagttactgcatgctagtataaaatcattcaggtttataatcttttcttaaatctgcaacatatggtcaactcttaaatgagtccttactgtgatctttgttttttatcgtgtttctttttcttctgctgcatcaggcaaatgttttaaacaagaccttgcttacccaagtcttggtgcctgttggactatacctggataaaggcacaaactgttggtaagcttagtagtctctatgtcatgttacttttagaactatctatgttgtctgttcatttgagtcagagtcagcaataaagacaatctaagttgatgtttcaatacttttttgtgtgatttggttggtgaattgacatgcaaaagcaccaggggtgcttgaaccaggatagcctgcgaaaaggcgggctatccgggaccaggctgagaaagtccctttgaacctgaacagggtaatgcctgcgcagggagtgtgcagttttttttttttcctgtagctttctaaaggagaagaagctactgttgccgctcgagtctcgttccacggttttcaacagttagtttcttatgagctaagagattcagcttaattggcttacagccataaaagaagtctttaactgatgcactaagtcactaacagtagggaataattcaatcaaaaaatcatccagattgataaaaatgcatttgcacctttggggcataagctgaaattactctgctcgcacaaatcagatttttaaaacttatgatcatgttttcaacttatactcgtttgtttacataatgggatgatcagttgttcactttgagcaaagcatactgggcaggtgtagggaaagtgaagctcatgaaactgatccactgtaattctcattgttcagcttttatttacacaaatgtgcgagtcaacacgtaaaagattatgatatattgcagtagaacataagtaaaacgaccttaccaaatgcgaaagaaagcttttcgagccggaatgatcaaaaggttacacattctgtactcattttcctgctatatgagatcacacaattatcaagagcctgaaactctggaataccaatatttagacacgttacatagagttttgaaggtttctcatttgaccagctcctgtgtttgacatgctttggtttcatatggttaatgtttctgctaaatcaaccatgagagatgtgaactatttctatatcatgtgtctgtaacgttggggccgcttgtgagcctcttccttcaccattttcagtttgatcagttaataacctcatgtaaaagcagtgagagagagaagagatcagagatggcacactgtttcctttggaaatcctgggcgttgttatg

SEQ ID NO: 5 - protein, 325 aa, Escherichia coli
MACGEFSLIARYFDRVRSSRLDVELGIGDDCALLNIPEKQTLAISTDTLVAGNHFLPDIDPADLAYKALAVNLSDLAAMGADPAWLTLALTLPDVDEAWLESFSDSLFDLLNYYDMQLIGGDTTRGPLSMTLGIHGFVPMGRALTRSGAKPGDWIYVTGTPGDSAAGLAILQNRLQVADAKDADYLIKRHLRPSPRILQGQALRDLANSAIDLSDGLISDLGHIVKASDCGARIDLALLPFSDALSRHVEPEQALRWALSGGEDYELCFTVPELNRGALDVALGHLGVPFTCIGQMTADIEGLCFIRDGEPVTLDWKGYDHFATP

SEQ ID NO: 6 - DNA, 978 bp, Escherichia coli
atggcatgtggcgagttctccctgattgcccgttattttgaccgtgtaagaagttctcgtcttgatgtcgaactgggcatcggcgacgattgcgcacttctcaatatccccgagaaacagaccctggcgatcagcactgatacgctggtggcgggtaaccatttcctccctgatatcgatcctgctgatctggcttataaagcactggcggtgaacctaagcgatctggcagcgatgggggccgatccggcctggctgacgctggcattaaccttaccggacgtagacgaagcgtggcttgagtccttcagcgacagtttgtttgatcttctcaattattacgatatgcaactcattggcggcgataccacgcgtgggccattatcaatgacgttgggtatccacggctttgttccgatgggacgagccttaacgcgctctggggcgaaaccgggtgactggatctatgtgaccggtacaccgggcgatagcgccgccgggctggcgattttgcaaaaccgtttgcaggttgccgatgctaaagatgcggactacttgatcaaacgtcatctccgtccatcgccgcgtattttacaggggcaggcactgcgcgatctggcaaattcagccatcgatctctctgacggtttgatttccgatctcgggcatatcgtgaaagccagcgactgcggcgcacgtattgacctggcattgctgccgttttctgatgcgctttctcgccatgttgaaccggaacaggcgctgcgctgggcgctctctggcggtgaagattacgagttgtgtttcactgtgccggaactgaaccgtggcgcgctggatgtggctctcggacacctgggcgtaccgtttacctgtatcgggcaaatgaccgccgatatcgaagggctttgttttattcgtgacggcgaacctgttacattagactggaaaggatatgaccattttgccacgccataa

SEQ ID NO: 7 - protein, 197 aa, Arabidopsis thaliana
MKKNLYFRYKPDVIKGDMDSIRRDVLDFYINLGTKVIDESHDQDTTDLDKCILYIRHSTLNQETSGLQILATGALGGRFDHEAGNLNVLYRYPDTRIVLLSDDCLIQLLPKTHRHEIHIQSSLEGPHCGLIPIGTPSAKTTTSGLQWDLSNTEMRFGGLISTSNLVKEEKITVESDSDLLWTISIKKTGLSIQDHTP

SEQ ID NO: 8 - DNA, 1770 bp, Arabidopsis thaliana
aattccttcttcttcttcttcttcttcttgtctccgatgtcagccatggatgttatgattcactcttcaagctttctcctcccttgcgacgaaactagtacagggacgagatacgctctcgttgttctgaaccagagtttgccacgattcactcctcttctctgggaacatggtactgatgaatcgctctattcttcagaatatatatgactctgttttcttggggttattctaaaattggtttcgaattttctgtgtagagcagcaaaacttcgtctctgtgctgatggaggcgctaatcgcatctacgacgaattgcctctcttcttccctaatgaagacgctttggccattcgaaacaggtccaactttctgaagctgtatctctcgctctcttactctttcttcggatttgaagattattattcaaatttagtgatggtttattggaataattagtctaaatttgagctggagatattttttttgtatgagttcatagattcaccttcttcttatctttatgtatattacatagagaaagatttcatgaagaagaacttgtattttaggtataagccggatgttatcaaaggagatatggattctatacgtcgtgacgtcctcgacttttatataaacttggtaagctcttagtctgtgatcacatttttagaattgttatccattagatcacaacctatgtgtgtggtgtctttgatagatctaacatgctttcgattgttctactttgtgtgtgtctttaatctctccagggaactaaggttatagatgaatctcatgatcaagacaccacagatcttgataaatgcattttgtatatccgtcactctactttgaatcaagagacttccggagtaagttttttttttattacagaggaatgctttctgcttctctttctctgcaactttttcaccttgtttttatgtttttcttctgtcagctccagattcttgccactggagcacttggaggaagatttgatcatgaagccggtaatctcaacgtcttatatcgatatccagatacaaggatagtccttttatctgacgattgcctcatccaactccttccaaagactcatcgacatgaaatacatattcagtcttctctagaagggcctcactgtggactcatacccattggaactccatctgctaaaaccacaacctcagggcttcaatgggatctcagtaagtaaacatcttgcatcatcatcattataatcgtcatcatcagcatcatctaactatagtttatatctgttctttttttataatcttcttaaggcaacactgagatgagatttggtgggttgataagtacatccaacttggttaaagaagagaaaatcacagtcgaatcggattcggatcttctctggactatatccatcaagaaaacaggactttccatacaagaccatacaccttaggcccggtaacaaaacacactttagtatattatacgctactatgatgttcctgatgcaacgaactagttcaacattattgtgttgatttgttgttcactgtactagctaataatacttgtaccattactgtcgttatacattaagatgcttttttctttggttctgtcattgtttatgtggggctttgttgatttgtcgtactcaaaattgtgactggatgttggttagatattggaatctacttgtgcggtatatgagaaaagacaaaattcaaaaggtgaatgactacgattgagcatatgtcatcaacc

SEQ ID NO: 9 - protein, 264 aa, Arabidopsis thaliana
MDVMIHSSSFLLPCDETSTGTRYALVVLNQSLPRFTPLLWEHAKLRLCADGGANRIYDELPLFFPNEDALAIRNRYKPDVIKGDMDSIRRDVLDFYINLGTKVIDESHDQDTTDLDKCILYIRHSTLNQETSGLQILATGALGGRFDHEAGNLNVLYRYPDTRIVLLSDDCLIQLLPKTHRHEIHIQSSLEGPHCGLIPIGTPSAKTTTSGLQWDLSNTEMRFGGLISTSNLVKEEKITVESDSDLLWTISIKKTGLSIQDHTP

SEQ ID NO: 10 - DNA, 2152 bp, Arabidopsis thaliana
cttttacaattgaaaatattttgtaataccatctctaaatctccctcttttgatctccaagtcctccatggttttagggcttttgctttgccgattgatacctaggctctgatttgatttttcctttagaaaactttcaaaaagtttcagtagattggcttaattggctctgttggtccttgtaaagaggataccttttcgtggttggaaaaaaagtcacgttttggcgaaacgctagtttaatgagatagagacgtggccatctttgaaaagtcaacgtttggtcgcaatcggtgacgtacactgtctatatttaatttttcagcaaccacagtgatttattgaatgaatcaatacataacaacaaaattatcagaaacacaattccttcttcttcttcttcttcttcttgtctccgatgtcagccatggatgttatgattcactcttcaagctttctcctcccttgcgacgaaactagtacagggacgagatacgctctcgttgttctgaaccagagtttgccacgattcactcctcttctctgggaacatggtactgatgaatcgctctattcttcagaatatatatgactctgttttcttggggttattctaaaattggtttcgaattttctgtgtagagcagcaaaacttcgtctctgtgctgatggaggcgctaatcgcatctacgacgaattgcctctcttcttccctaatgaagacgctttggccattcgaaacaggtccaactttctgaagctgtatctctcgctctcttactctttcttcggatttgaagattattattcaaatttagtgatggtttattggaataattagtctaaatttgagctggagatattttttttgtatgagttcatagattcaccttcttcttatctttatgtatattacatagagaaagatttcatgaagaagaacttgtattttaggtataagccggatgttatcaaaggagatatggattctatacgtcgtgacgtcctcgacttttatataaacttggtaagctcttagtctgtgatcacatttttagaattgttatccattagatcacaacctatgtgtgtggtgtctttgatagatctaacatgctttcgattgttctactttgtgtgtgtctttaatctctccagggaactaaggttatagatgaatctcatgatcaagacaccacagatcttgataaatgcattttgtatatccgtcactctactttgaatcaagagacttccggagtaagttttttttttattacagaggaatgctttctgcttctctttctctgcaactttttcaccttgtttttatgtttttcttctgtcagctccagattcttgccactggagcacttggaggaagatttgatcatgaagccggtaatctcaacgtcttatatcgatatccagatacaaggatagtccttttatctgacgattgcctcatccaactccttccaaagactcatcgacatgaaatacatattcagtcttctctagaagggcctcactgtggactcatacccattggaactccatctgctaaaaccacaacctcagggcttcaatgggatctcagtaagtaaacatcttgcatcatcatcattataatcgtcatcatcagcatcatctaactatagtttatatctgttctttttttataatcttcttaaggcaacactgagatgagatttggtgggttgataagtacatccaacttggttaaagaagagaaaatcacagtcgaatcggattcggatcttctctggactatatccatcaagaaaacaggactttccatacaagaccatacaccttaggcccggtaacaaaacacactttagtatattatacgctactatgatgttcctgatgcaacgaactagttcaacattattgtgttgatttgttgttcactgtactagctaataatacttgtaccattactgtcgttatacattaagatgcttttttctttggttctgtcattgtttatgtggggctttgttgatttgtcgtactcaaaattgtgactggatgttggttagatattggaatctacttgtgcggtatatgagaaaagacaaaattcaaaaggtgaatgactacgattgagcatatgtcatcaacc

SEQ ID NO: 11 - protein, 267 aa, Arabidopsis thaliana
MSAMDVMIHSSSFLLPCDETSTGTRYALVVLNQSLPRFTPLLWEHAKLRLCADGGANRIYDELPLFFPNEDALAIRNRYKPDVIKGDMDSIRRDVLDFYINLGTKVIDESHDQDTTDLDKCILYIRHSTLNQETSGLQILATGALGGRFDHEAGNLNVLYRYPDTRIVLLSDDCLIQLLPKTHRHEIHIQSSLEGPHCGLIPIGTPSAKTTTSGLQWDLSNTEMRFGGLISTSNLVKEEKITVESDSDLLWTISIKKTGLSIQDHTP

SEQ ID NO: 12 - DNA, 1788 bp, Arabidopsis thaliana
caaaattatcagaaacacaattccttcttcttcttcttcttcttcttgtctccgatgtcagccatggatgttatgattcactcttcaagctttctcctcccttgcgacgaaactagtacagggacgagatacgctctcgttgttctgaaccagagtttgccacgattcactcctcttctctgggaacatggtactgatgaatcgctctattcttcagaatatatatgactctgttttcttggggttattctaaaattggtttcgaattttctgtgtagagcagcaaaacttcgtctctgtgctgatggaggcgctaatcgcatctacgacgaattgcctctcttcttccctaatgaagacgctttggccattcgaaacaggtccaactttctgaagctgtatctctcgctctcttactctttcttcggatttgaagattattattcaaatttagtgatggtttattggaataattagtctaaatttgagctggagatattttttttgtatgagttcatagattcaccttcttcttatctttatgtatattacatagagaaagatttcatgaagaagaacttgtattttaggtataagccggatgttatcaaaggagatatggattctatacgtcgtgacgtcctcgacttttatataaacttggtaagctcttagtctgtgatcacatttttagaattgttatccattagatcacaacctatgtgtgtggtgtctttgatagatctaacatgctttcgattgttctactttgtgtgtgtctttaatctctccagggaactaaggttatagatgaatctcatgatcaagacaccacagatcttgataaatgcattttgtatatccgtcactctactttgaatcaagagacttccggagtaagttttttttttattacagaggaatgctttctgcttctctttctctgcaactttttcaccttgtttttatgtttttcttctgtcagctccagattcttgccactggagcacttggaggaagatttgatcatgaagccggtaatctcaacgtcttatatcgatatccagatacaaggatagtccttttatctgacgattgcctcatccaactccttccaaagactcatcgacatgaaatacatattcagtcttctctagaagggcctcactgtggactcatacccattggaactccatctgctaaaaccacaacctcagggcttcaatgggatctcagtaagtaaacatcttgcatcatcatcattataatcgtcatcatcagcatcatctaactatagtttatatctgttctttttttataatcttcttaaggcaacactgagatgagatttggtgggttgataagtacatccaacttggttaaagaagagaaaatcacagtcgaatcggattcggatcttctctggactatatccatcaagaaaacaggactttccatacaagaccatacaccttaggcccggtaacaaaacacactttagtatattatacgctactatgatgttcctgatgcaacgaactagttcaacattattgtgttgatttgttgttcactgtactagctaataatacttgtaccattactgtcgttatacattaagatgcttttttctttggttctgtcattgtttatgtggggctttgttgatttgtcgtactcaaaattgtgactggatgttggttagatattggaatctacttgtgcggtatatgagaaaagacaaaattcaaaaggtgaatgactacgattgagcatatgtcatcaacc

SEQ ID NO: 13 - protein, 180 aa, Arabidopsis thaliana
MDSIRRDVLDFYINLGTKVIDESHDQDTTDLDKCILYIRHSTLNQETSGLQILATGALGGRFDHEAGNLNVLYRYPDTRIVLLSDDCLIQLLPKTHRHEIHIQSSLEGPHCGLIPIGTPSAKTTTSGLQWDLSNTEMRFGGLISTSNLVKEEKITVESDSDLLWTISIKKTGLSIQDHTP

SEQ ID NO: 14 - DNA, 1781 bp, Arabidopsis thaliana
atcagaaacacaattccttcttcttcttcttcttcttcttgtctccgatgtcagccatggatgttatgattcactcttcaagctttctcctcccttgcgacgaaactagtacagggacgagatacgctctcgttgttctgaaccagagtttgccacgattcactcctcttctctgggaacatggtactgatgaatcgctctattcttcagaatatatatgactctgttttcttggggttattctaaaattggtttcgaattttctgtgtagagcagcaaaacttcgtctctgtgctgatggaggcgctaatcgcatctacgacgaattgcctctcttcttccctaatgaagacgctttggccattcgaaacaggtccaactttctgaagctgtatctctcgctctcttactctttcttcggatttgaagattattattcaaatttagtgatggtttattggaataattagtctaaatttgagctggagatattttttttgtatgagttcatagattcaccttcttcttatctttatgtatattacatagagaaagatttcatgaagaagaacttgtattttaggtataagccggatgttatcaaaggagatatggattctatacgtcgtgacgtcctcgacttttatataaacttggtaagctcttagtctgtgatcacatttttagaattgttatccattagatcacaacctatgtgtgtggtgtctttgatagatctaacatgctttcgattgttctactttgtgtgtgtctttaatctctccagggaactaaggttatagatgaatctcatgatcaagacaccacagatcttgataaatgcattttgtatatccgtcactctactttgaatcaagagacttccggagtaagttttttttttattacagaggaatgctttctgcttctctttctctgcaactttttcaccttgtttttatgtttttcttctgtcagctccagattcttgccactggagcacttggaggaagatttgatcatgaagccggtaatctcaacgtcttatatcgatatccagatacaaggatagtccttttatctgacgattgcctcatccaactccttccaaagactcatcgacatgaaatacatattcagtcttctctagaagggcctcactgtggactcatacccattggaactccatctgctaaaaccacaacctcagggcttcaatgggatctcagtaagtaaacatcttgcatcatcatcattataatcgtcatcatcagcatcatctaactatagtttatatctgttctttttttataatcttcttaaggcaacactgagatgagatttggtgggttgataagtacatccaacttggttaaagaagagaaaatcacagtcgaatcggattcggatcttctctggactatatccatcaagaaaacaggactttccatacaagaccatacaccttaggcccggtaacaaaacacactttagtatattatacgctactatgatgttcctgatgcaacgaactagttcaacattattgtgttgatttgttgttcactgtactagctaataatacttgtaccattactgtcgttatacattaagatgcttttttctttggttctgtcattgtttatgtggggctttgttgatttgtcgtactcaaaattgtgactggatgttggttagatattggaatctacttgtgcggtatatgagaaaagacaaaattcaaaaggtgaatgactacgattgagcatatgtcatcaacc

SEQ ID NO: 15 - protein, 265 aa, Arabidopsis thaliana
MLSAMDVMIHSSSFLLPCDETCGTRYALVVLNQNLPRFTPLLWEHAKLRLCADGGANRIYDELPLFFPHEDPFVIRNRYKPDVIKGDMDSIRRDVLDFYVYWGTKVIDESHDQDTTDLDKCISYIRHSTLNQESSRILATGALGGRFDHEAGNLNVLYRYPDTRIVLLSDDCLIQLLPKTHRHEIHIHSSLQGPHCGLIPIGTPSANTTTSGLKWDLSNTEMRFGGLISTSNLVKEEIITVESDSDLLWTISIKKTGLPVQDHKP

SEQ ID NO: 16 - DNA, 1450 bp, Arabidopsis thaliana
ggcggaaacttacgccggcggcacaaactttgtgtcttgctcaagatgattgcgtcgaattttatgtaaacaattggctctccgatgttgtcagccatggatgttatgattcactcttcaagctttctcctcccttgcgacgaaacttgtgggacgagatacgctctcgttgttcttaaccagaatttaccacgattcactcctcttctctgggaacatggtaattatctctctattcttcagaatatataaatgaatctgttttttttttattacgaatattctgtgtagaacagcaaaacttcgtctctgtgctgatggaggcgctaatcgcatctacgacgaattacctctcttcttccctcacgaagacccttttgtcattcgaaacaggtctcagatttatgtctctatttctctcatagagaaaggtttcaggaagaagaagttgaaactggaaagttttgtctttttattttatagatagttctgagaattgtactttttaggtataagcccgatgttatcaagggagatatggattctatacgccgtgacgtcctcgacttttatgtttactgggtaagctctttcttcttactattattatgcattagatcacaacagtgtctgtatttgatctaacatgctttttctccagggaactaaggttatagatgaatctcatgatcaagataccactgatcttgataaatgcatttcgtatatccgtcactctactttgaatcaggagagttccagagtaagttcttttttttaattacagaggaatgctagctcgctgcgtttgtttctctgcaacattttcactatctgttttatgttagctccagattcttgccactggagcactcgggggaagattcgatcatgaagccggtaatctcaacgtcttatatcgatatccagacacaaggatagtccttttatctgatgattgtctcatccaactccttccaaagactcatcgacatgaaatacatattcactcttctcttcaaggacctcactgtggacttatacccattggaactccatctgccaataccactacctcagggcttaaatgggatctcagtaagtaacatcttccatcatcatcatcatcatcattatcagatcgatctaattattctggtttttttttataatcgtcttacaggcaacactgagatgagatttggtgggttgataagtacatcgaacttggttaaagaagagataatcacagtcgaatcggattcggatcttctctggactatttccatcaagaagacaggacttcctgtacaagaccataaaccttagtggcgccactctttagttttatacgctactattatattcatgatgcatcgaagaattacttcaacattattgtgttgatttgttttcactataccagcaaataacaatttattgccttacc

SEQ ID NO: 17 - protein, 267 aa, Arabidopsis thaliana
MLSAMDVMIHSSSFLLPCDETCGTRYALVVLNQNLPRFTPLLWEHAKLRLCADGGANRIYDELPLFFPHEDPFVIRNRYKPDVIKGDMDSIRRDVLDFYVYWGTKVIDESHDQDTTDLDKCISYIRHSTLNQESSRLQILATGALGGRFDHEAGNLNVLYRYPDTRIVLLSDDCLIQLLPKTHRHEIHIHSSLQGPHCGLIPIGTPSANTTTSGLKWDLSNTEMRFGGLISTSNLVKEEIITVESDSDLLWTISIKKTGLPVQDHKP

SEQ ID NO: 18 - DNA, 1450 bp, Arabidopsis thaliana; identical to SEQ ID NO: 16

SEQ ID NO: 19 - DNA, 33 bp, Artificial Sequence
  Synthetic primer
aataaactagtaaggtcagtatgtttagtctgt

SEQ ID NO: 20 - DNA, 33 bp, Artificial Sequence
  Synthetic primer
aataagtcgaccataacaacgcccaggatttcc

SEQ ID NO: 21 - DNA, 32 bp, Artificial Sequence
  Synthetic primer
aaaaactgcacaccccctgcgcaggcattacc

SEQ ID NO: 22 - DNA, 24 bp, Artificial Sequence
  Synthetic primer
ccatcttttgaagaatgctttcct

SEQ ID NO: 23 - DNA, 22 bp, Artificial Sequence
  Synthetic primer
gaacacgacgaaagggaacttt

SEQ ID NO: 24 - DNA, 25 bp, Artificial Sequence
  Synthetic primer
gcctgttggactatacctggataaa

SEQ ID NO: 25 - DNA, 31 bp, Artificial Sequence
  Synthetic primer
tgactcaaatgaacagacaacatagatagtt

SEQ ID NO: 26 - DNA, 23 bp, Artificial Sequence
  Synthetic primer
cttggtgcctgttggactatacc

SEQ ID NO: 27 - DNA, 23 bp, Artificial Sequence
  Synthetic primer
tcaggttcaaagggactttctca

SEQ ID NO: 28 - DNA, 22 bp, Artificial Sequence
  Synthetic primer
tagcattgatggctcatcctga

SEQ ID NO: 29 - DNA, 21 bp, Artificial Sequence
  Synthetic primer
ttgtgccattgaattgaaccc

SEQ ID NO: 30 - DNA, 20 bp, Artificial Sequence
  Synthetic primer
ctacgacgccgaggtcaaga

SEQ ID NO: 31 - DNA, 20 bp, Artificial Sequence
  Synthetic primer
cgttgtgggaggtgatgtcc

SEQ ID NO: 32 - DNA, 19 bp, Artificial Sequence
  Synthetic primer
gcatcgagctgaagggcat

SEQ ID NO: 33 - DNA, 22 bp, Artificial Sequence
  Synthetic primer
tcggccatgatatagacgttgt

SEQ ID NO: 34 - DNA, 27 bp, Artificial Sequence
  Synthetic primer
tttcttctccttctagtgaatcaaaca

SEQ ID NO: 35 - DNA, 33 bp, Artificial Sequence
  Synthetic primer
gtcgacagctggagacaaacgaaaatatgaatc

SEQ ID NO: 36 - DNA, 34 bp, Artificial Sequence
  Synthetic primer
aataaccatggctgcttcagtacactgtaccttg

SEQ ID NO: 37 - DNA, 35 bp, Artificial Sequence
  Synthetic primer
aatagctagcttatttctgagcagctttgacatag

SEQ ID NO: 38 - DNA, 21 bp, Artificial Sequence
  Synthetic primer
gcttcaatgggatctcagcaa

SEQ ID NO: 39 - DNA, 21 bp, Artificial Sequence
  Synthetic primer
cgaatccgattcgactgtgat

SEQ ID NO: 40 - DNA, 22 bp, Artificial Sequence
  Synthetic primer
tgggatctcagcaacactgaga

SEQ ID NO: 41 - DNA, 20 bp, Artificial Sequence
  Synthetic primer
gaagatccgaatccgattcg

SEQ ID NO: 42 - DNA, 22 bp, Artificial Sequence
  Synthetic primer
cgctattgtgaggttgaccaga

SEQ ID NO: 43 - DNA, 21 bp, Artificial Sequence
  Synthetic primer
caaaagttggtcccattctcg

SEQ ID NO: 44 - DNA, 22 bp, Artificial Sequence
  Synthetic primer
ggccaccatcatacaactgagg

SEQ ID NO: 45 - DNA, 20 bp, Artificial Sequence
  Synthetic primer
actaactccatgggaccggc